(19) United States
(12) Patent Application Publication
(10) Pub. No.: US 2024/0175050 A1
(43) Pub. Date: May 30, 2024

(54) METHODS AND MATERIALS FOR TREATING PROPIONIC ACIDEMIA

(71) Applicant: Mayo Foundation for Medical Education and Research, Rochester, MN (US)

(72) Inventor: Michael A. Barry, Rochester, MN (US)

(21) Appl. No.: 18/282,645

(22) PCT Filed: Mar. 18, 2022
(86) PCT No.: PCT/US2022/021003

§ 371 Date: Sep. 18, 2023

Related U.S. Application Data
(60) Provisional application No. 63/163,198, filed on Mar. 19, 2021.

(51) Int. Cl. C12N 15/86; A61K 48/00; A61P 3/00; C07K 14/47; C12N 9/00

(52) U.S. Cl. CPC C12N 15/86; A61K 48/0058; A61P 3/00; C07K 14/47; C12N 9/93; C12N 2750/14143; C12N 2830/008; C12Y 604/01003

(57) ABSTRACT

Methods and materials for treating propionic acidemia are provided herein. For example, methods and materials for using AAVs to express a PCCA polypeptide and/or a PCCB polypeptide within a mammal to treat propionic acidemia are provided.

### CROSS-REFERENCE TO RELATED APPLICATIONS

This application claims benefit of priority from U.S. Provisional Application No. 63/163,198, filed on Mar. 19, 2021. The disclosure of the prior application is considered part of (and is incorporated by reference in) the disclosure of this application.

### SEQUENCE LISTING

This application contains a Sequence Listing that has been submitted electronically as an ASCII text file named SequenceListing.txt. The ASCII text file, created on Mar. 14, 2022, is 41.1 kilobytes in size. The material in the ASCII text file is hereby incorporated by reference in its entirety.

### TECHNICAL FIELD

This document relates to methods and materials for treating propionic acidemia. For example, this document provides methods and materials for using adeno-associated viruses (AAVs), such as AAVs designed to express codon-optimized cDNA encoding propionyl-CoA carboxylase (PCC) A (PCCA) and/or B (PCCB), to treat mammals with propionic acidemia.

### BACKGROUND

Propionic acidemia (PA) is a rare but highly morbid disease affecting about 1 in 100,000 live births, although specific populations with founder mutations exist in which the incidence of PA can be as high as 1 in 1,000 (Childs et al., Pediatrics 1961, 27:522-538; and Ravn et al., Am J Hum Genet 2000, 67(1):203-206). PA is caused by mutations in the PCCA or PCCB genes that encode the A and B subunits of the mitochondrial PCC enzyme (Huang et al., Nature 2010, 466(7309): 1001-1005), which catalyzes the conversion of propionyl-CoA to methylmalonyl-CoA. Functional PCC is formed as a dodecamer of six subunits of PCCA and six subunits of PCCB. Mutations in PCCA can result in loss or reduced levels of the PCCA subunit, and mutations in PCCB can result in loss or reduced levels of the PCCB subunit. A reduction in expression of either subunit can disrupt the ability to form functional dodecamer forms of the PCC enzyme. In addition, imbalanced expression of either subunit can lead to degradation of the other subunit.

The primary substrates of PCC are cholesterol, valine, odd-chain fatty acids, methionine, isoleucine, threonine, and methionine (C-VOMIT), and propionate produced by gut bacteria (Thompson et al., Metabolism 1990, 39(11): 1133-1137; Leonard and Bodamer, Eur J Pediatr 1997, 156 (Suppl 1):S88-89; and Bain et al., Lancet 1988, 1(8594): 1078-1079). Increased consumption or production of any of these substrates can exacerbate the symptoms of PA and provoke metabolic decompensation and hyperammonemia (Grier et al., Pediatr Res 1981, 15:562).

Decreased PCC activity in PA patients leads to accumulation of propionyl-CoA and elevated levels of propionylcarnitine, tiglyglycine (Rasmussen et al., J Pediatr 1972, 81(5):970-972), propionylglycine (Rasmussen et al., Clin Sci 1972, 42(6):665-671), and organic acids such as methylcitrate (Lehnert et al., Eur J Pediatr 1994, 153(7 Suppl 1):S68-80) in blood and urine, either as a direct consequence of elevated propionyl-CoA (e.g., propionylcarnitine) or due to aberrant metabolism of propionate (FIG. 1). Propionylcarnitine is thought to be produced as an aberrant product to shunt propionate out of the cell as it accumulates to excessive levels. Propionyl-CoA also can drive synthesis of odd chain fatty acids (C15:0, C17:0, and C17:1) (FIG. 1), and the detection of these odd chain fatty acids in the plasma of patients seems to correlate with clinical severity (Lynen, Fed Proc 1961, 20:941-951; and Sperl et al., Eur J Pediatr 2000, 159(1-2):54-58). PCC deficiency has the potential to affect many organ systems and can lead to development of potentially fatal cardiomyopathy. Heart-related symptoms and effects of PA can include, for example, elevated levels of cardiac B-type natriuretic peptide (BNP) transcripts, elevated cardiac and plasma triglycerides, accumulation of lipid droplets within cardiac tissue, cardiac hypertrophy, thinning of left ventricle (LV) walls, and increased internal chamber diameter that can indicate dilated cardiomyopathy.

### SUMMARY

This document provides methods and materials for treating a mammal (e.g., a human) having PA. For example, DNA virus vectors (e.g., AAVs) designed to express one or more PCC polypeptides (e.g., a PCCA polypeptide and/or a PCCB polypeptide) can be delivered to a mammal (e.g., a human) having PA to treat the mammal. In some cases, DNA virus vectors (e.g., AAVs) designed to express one or more PCC polypeptides (e.g., a PCCA polypeptide and/or a PCCB polypeptide) can be delivered to cells within a particular organ or tissue (e.g., the liver, kidneys, brain, or muscle) of a mammal (e.g., a human) having PA. As discussed herein, using AAVs to deliver a PCCA polypeptide and/or a PCCB polypeptide to a mammal can reduce one or more symptoms or effects of PA in the mammal (e.g., reduce levels of cardiac BNP transcripts, reduce cardiac and/or plasma triglyceride levels, reduce the amount of lipid droplets within cardiac tissue, reduce cardiac hypertrophy, reduce the thinning of LV walls, reduce the internal chamber diameter, and/or reduce the development, progression, or likelihood of cardiomyopathy).

In a first aspect, this document provides nucleic acid constructs (e.g., adeno-associated virus serotype rh10 (AAVrh10) nucleic acid constructs. The nucleic acid constructs can include, consist of, or consist essentially of a nucleotide sequence encoding a PCCA polypeptide or a PCCB polypeptide, and a Cbh promoter operably linked to the nucleotide sequence encoding the PCCA polypeptide or the PCCB polypeptide. The nucleotide sequence encoding the PCCA polypeptide or the PCCB polypeptide can be codon optimized for expression in humans. The nucleotide sequence can encode a PCCA polypeptide containing the sequence set forth in SEQ ID NO:3 or SEQ ID NO:4, or a sequence at least 95% identical to SEQ ID NO:3 or SEQ ID NO:4. The nucleotide sequence can encode a PCCB polypeptide containing the sequence set forth in SEQ ID NO:6, or a sequence at least 95% identical to SEQ ID NO:6. The construct can further contain a nucleotide sequence encoding a Rad23 polypeptide or a portion thereof.

In another aspect, this document features an AAV nucleic acid construct that includes, consists essentially of, or consists of: a first nucleotide sequence encoding a PCCA polypeptide, a second nucleotide sequence encoding a PCCB polypeptide, and a promoter operably linked to the first nucleotide sequence encoding the PCCA polypeptide. The first nucleotide sequence encoding the PCCA polypeptide and the second nucleotide sequence encoding the PCCB polypeptide can be codon optimized for expression in humans. The first nucleotide sequence can encode a PCCA polypeptide containing the sequence set forth in SEQ ID NO:3 or SEQ ID NO:4, or a sequence at least 95% identical to SEQ ID NO:3 or SEQ ID NO:4, and the second nucleotide sequence can encode a PCCB polypeptide containing the sequence set forth in SEQ ID NO:6, or a sequence at least 95% identical to SEQ ID NO:6. The AAV can be AAV1, AAV8, AAV9, or AAVrh10. The AAV nucleic acid construct can further include a nucleotide sequence encoding a Rad23 polypeptide or a portion thereof.

In another aspect, this document features a composition. The composition can contain, consist essentially of, or consist of: (a) a first AAV nucleic acid construct containing a first promoter operably linked to a nucleotide sequence encoding a PCCA polypeptide or a PCCB polypeptide, wherein the first AAV nucleic acid construct is an AAVrh10 nucleic acid construct, and (b) a second AAV nucleic acid containing a first promoter operably linked to a nucleotide sequence encoding a PCCA polypeptide or a PCCB polypeptide, wherein the second AAV nucleic acid construct is not an AAVrh10 nucleic acid construct. The second AAV nucleic acid construct can be an AAV1 nucleic acid construct or an AAV8 nucleic acid construct. The first promoter can be a Cbh promoter, the second promoter can be a Cbh promoter, or both the first promoter and the second promoter can be Cbh promoters. The first or second AAV nucleic acid construct can include a nucleotide sequence encoding a PCCA polypeptide, where the PCCA polypeptide contains the sequence set forth in SEQ ID NO:3 or SEQ ID NO:4, or a sequence at least 95% identical to SEQ ID NO:3 or SEQ ID NO:4. The first or second AAV nucleic acid construct can include a nucleotide sequence encoding a PCCB polypeptide, where the PCCB polypeptide includes the sequence set forth in SEQ ID NO:6, or a sequence at least 95% identical to SEQ ID NO:6. The first AAV nucleic acid can further include a nucleotide sequence encoding a Rad23 polypeptide or a portion thereof, the second AAV nucleic acid can further include a nucleotide sequence encoding a Rad23 polypeptide or a portion thereof, or both the first AAV nucleic acid and the second AAV nucleic acid can further include a nucleotide sequence encoding a Rad23 polypeptide or a portion thereof.

In still another aspect, this document features a method for treating a mammal having PA. The method can include, or consist essentially of administering to the mammal an AAVrh10 nucleic acid construct provided herein, where the administering results in an increased level of a PCCA polypeptide and/or a PCCB polypeptide in the mammal.

This document also features a method for treating a mammal having PA, where the method includes, or consists essentially of, administering to the mammal an AAV nucleic acid construct provided herein, where the administering results in an increased level of a PCCA polypeptide and/or a PCCB polypeptide in the mammal.

In another aspect, this document features a method for treating a mammal having PA, where the method includes, or consists essentially of, administering to the mammal a composition provided herein, where the administering results in an increased level of a PCCA polypeptide and/or a PCCB polypeptide in the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing metabolic pathways involved in propionic acidemia. Cholesterol, odd-chain fatty acids, valine, methionine, isoleucine, and threonine (so-called “C-VOMIT” substrates) are the major dietary sources that contribute to the total pool of propionyl-CoA. Decreased activity of propionyl-CoA carboxylase in PA leads to accumulation of propionyl-CoA, which also results in elevated propionylcarnitine and methylcitrate levels. Propionyl-CoA can serve as a primer for fatty acid synthesis (inset), resulting in higher than normal levels of odd chain fatty acids.

FIG. 2A shows a representative human PCCA nucleic acid sequence with a long mitochondrial leader (SEQ ID NO:1). FIG. 2B shows a representative human PCCA nucleic acid sequence with a short mitochondrial leader (SEQ ID NO:2). FIG. 2C shows a representative human PCCA amino acid sequence (SEQ ID NO:3) encoded by SEQ ID NO:1, and FIG. 2D shows a representative human PCCA amino acid sequence (SEQ ID NO:4) encoded by SEQ ID NO:2.

FIG. 3A shows a representative human PCCB nucleic acid sequence (SEQ ID NO:5), and FIG. 3B shows a representative human PCCB amino acid sequence (SEQ ID NO:6) encoded by SEQ ID NO:5.

FIG. 4A is a diagram illustrating an AAVrh10-Cbh-PCCA vector, which is an AAVrh10 vector containing a PCCA coding sequence coupled to a Cbh promoter (a CMV early enhancer fused to modified chicken B-actin promoter). The vector also includes, as indicated, AAV2 left and right inverted terminal repeats, and a human growth hormone (hGH) poly A sequence. This AAV genome can be propagated in a plasmid with ampicillin or kanamycin resistance, with and without insertions in the plasmid backbone to reduce reverse packaging. FIGS. 4B and 4C show two nucleotide sequences (SEQ ID NOS:7 and 8) for the AAVrh10-Cbh-PCCA vector (independent of the serotype used to package the vector).

FIG. 5 is a diagram depicting nucleic acid constructs designed to express both PCCA and PCCB polypeptides. The constructs illustrated in FIG. 5 contain a variety of enhancers, promoters, polyA sequences, and minimal polyadenylation signals, as well as optional mitochondrial processing peptidase (MPP) and/or self-cleaving peptide (P2A) sequences.

FIG. 6 is an image showing the results of western blotting of the indicated mouse and human tissues for PCCA.

FIG. 7A includes a pair of images showing increased brain inflammation in A138T mice vs. wild type mice, as evidenced by increased numbers of astrocytes detected by staining for Glial Fibrillary Acidic Protein (GFAP) in the thalamic region of A138T mice. FIG. 7B is a graph plotting novel object recognition in wild type and A138T mice, showing diminished cognition/learning in A138T mice as indicated by loss of novel object recognition after training.

FIG. 8A includes images showing neutral lipids stained with oil red in liver samples from wild type and A138T mice (top), as well as images showing fat deposits in the liver of an A138T mouse (bottom left) vs. a wild type mouse (bottom right). FIG. 8B includes a pair of images showing liver structures in samples from an A138T mouse. Circles in the top image indicate multi-mitochondrial bodies. Arrows in the lower image indicate areas of endoplasmic reticulum (ER) dilation. The images also show overall decreased mitochondria-ER interaction. FIG. 8C includes a pair of images showing liver samples from a wild type mouse (left) and an A138T mouse, assessed with liver imaging using 2 photon microscopy with coherent anti-strokes spectroscopy (CARS) to detect lipids and fatty liver.

FIG. 9A depicts the relative positions of the UbL and UBA domains within a Rad23 polypeptide. FIG. 9B is a schematic showing the structures of several nucleic acids encoding PCCA and/or PCCB polypeptides in combination with a Rad23 polypeptide or a fragment thereof. FIG. 9C shows a representative human Rad23 nucleic acid sequence (SEQ ID NO:9), and FIG. 9D shows a representative human Rad23 amino acid sequence (SEQ ID NO:10) encoded by SEQ ID NO:9.

FIGS. 10A-10C are graphs showing biochemical changes in cardiac tissue in the Pcca^{-/-}(A138T) mouse model of PA. Intracardiac propionylcarnitine levels were analyzed in 9-month-old mice (n=8 each group) by homogenizing 20 mg of heart tissue in water and using a tandem mass spectrometry method for newborn screening (FIG. 10A). Triglyceride levels were measured in plasma (FIG. 10B) and cardiac tissue (FIG. 10C) from wild type and Pcca^{-/-}(A138T) mice about 9-months of age. The open triangle and diamond symbols in the graphs correspond to transmission electron microscopy (TEM) images presented in FIG. 11.

FIG. 11 is a series of representative images from TEM of cardiac tissue. Cardiac tissue from about 9-month-old wild type (top row), and Pcca^{-/-}(A138T) mice (bottom 3 rows) was analyzed by TEM. Panels in the left column were viewed at 5,000× magnification (black bars represent 5 μm). Open white boxes correspond to the right column of panels at 20,000× magnification (black bars represent 1 μm). The open diamond and triangle symbols correspond to specific data points from these mice presented in FIGS. 10B and 10C.

FIGS. 12A-12E are a series of graphs plotting structural changes in cardiac tissue from wild type and Pcca^{-/-}(A138T) mice. The average mitochondrial area was analyzed based on TEM images of mouse heart (n=3 mice, at least 100 mitochondrial tracings per mouse) using ImageJ software (FIG. 12A). Echocardiography also was performed on adult male wild type and Pcca^{-/-}(A138T) mice (n=6 each) for the following parameters: ejection fraction (FIG. 12B), left ventricular posterior wall thickness during diastole (LVPWd) (FIG. 12C), intraventricular septum thickness during diastole (IVSd) (FIG. 12D), and left ventricle internal diameter (LVIDd) divided by body mass (FIG. 12E). Error bars depict SEM. * p<0.05, **** p<0.0001 by student t-test. FIG. 12F is a graph plotting heart mass as a percentage of total body mass in wild type and A138T (PA) mice fed a diet containing 45% fat or 90% fat.

FIGS. 13A and 13B are a pair of graphs plotting PCCA activity in cardiac tissue 1.5 years after injecting mice with 5×10¹¹ viral genomes (vg) of AAV8-CMV-PCCACo. Results are plotted across both genders (FIG. 13A) or by male vs. female (FIG. 13B).

FIGS. 14A-14F are a series of graphs plotting data from cardiac analysis of Pcca^{-/-}(A138T) mice treated with tissue-specific gene therapy vectors. Male Pcca^{-/-} (A138T) mice were intravenously administered 5×10¹¹ vg of either AAV8-TTR-PCCA or AAV1-MCK6-PCCA by tail vein injection at 5-weeks of age. Echocardiography was performed about 8 months later alongside untreated and age-matched male Pcca^{-/-} (A138T) mice (n=10 for untreated mice, n=5 for treatment groups) for the following parameters: ejection fraction (FIG. 14A), LVIDd divided by body mass (FIG. 14B), LVPWd (FIG. 14C), and IVSd (FIG. 14D). After completion of echocardiography, the animals were euthanized and heart mass was measured and indexed to body mass (FIG. 14E). Levels of BNP mRNA transcripts were analyzed by RT-qPCR in male wild type, untreated Pcca^{-/-}(A138T) (n=8 each), AAV8-TTR-PCCA treated, and AAV1-MCK6-PCCA treated (n=5 each) mice (FIG. 14F). Error bars depict SEM. * p<0.05, ** p<0.01, by one-way ANOVA.

FIG. 15 is a graph plotting methyl citrate levels in A138T mice after intravenous administration of AAV8-CMV-hPCCA at varied doses as compared to mice injected with negative control AAV8-GL expressing GFP-Luciferase. These are compared to mice that received an intravenous administration of a combination AAV8-hPCCA and AAV8-hPCCB vectors.

FIGS. 16A and 16B are graphs plotting C3 (propionylcarnitine) levels in male (FIG. 16A) and female (FIG. 16B) mice over an 8-week period of time after an intravenous administration of an AAVrh10-RSV-PCCA-PCCB vector.

FIGS. 17A and 17B are graphs showing a comparison of the effects of broadly expressing AAV10-Cbh-PCCA vs. muscle-specific expressing AAVrh10-MCK-PCCA on blood propionyl carnitine (C3) levels. Pcca−/−A138T mice were injected intravenously with PBS or 5×10¹¹ viral genomes of the indicated vectors, and C3 levels were measured on the indicated weeks after treatment. FIG. 17A is a graph plotting C3 levels over time. FIG. 17B is a graph plotting C3 levels on week 8.

FIGS. 18A and 18B are images of western blots showing a comparison of expression levels for PCCA and PCCB single or fusion proteins, expressed from coding sequences in different arrangements within expression constructs, with and without modified mitochondrial targeting sequences and P2A elements. FIG. 18A is an image of a western blot showing a comparison of single codon-optimized PCCA and PCCB expression plasmids with Cbh or CMV promotors to PCCA-PCCB or PCCB-PCCA fusion protein expression vectors with RSV or CMV promoters. FIG. 18B is an image of a western blot showing a comparison of different PCCA-PCCB and PCCB-PCCA fusion protein vectors with and without native or wobbled (MTB) mitochondrial targeting sequences and with or without a P2A element before the second MTB targeting sequence. Also shown is expression from RSV, PGKi, and CMV promoters.

FIG. 19 is a diagram depicting an example of an AAV vector that can express both PCCA and PCCB.

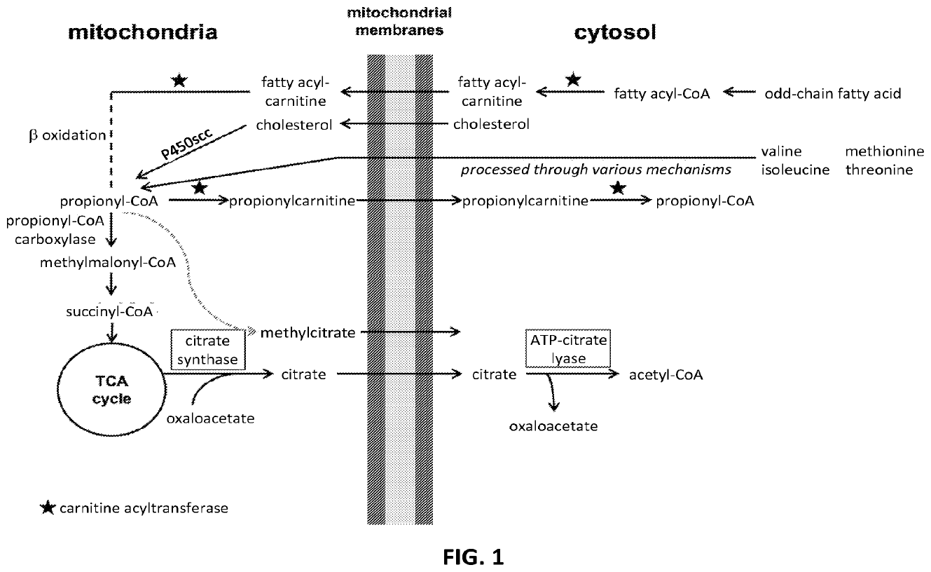

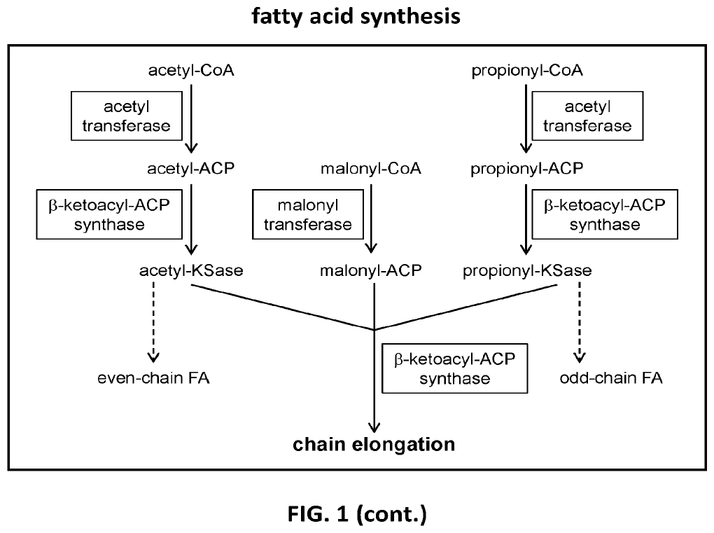

### DETAILED DESCRIPTION

This document provides methods and materials for treating a mammal (e.g., a human) having PA. For example, this document provides DNA virus nucleic acids (e.g., AAV vectors) designed to express a PCCA polypeptide and/or a PCCB polypeptide that can be delivered to a mammal (e.g., a human) having PA in order to treat the mammal. In some cases, AAVs designed to express a PCCA polypeptide and/or a PCCB polypeptide can be delivered to cells within a mammal (e.g., a human) having PA in the form of an AAVrh10 viral vector. In some cases, AAVs designed to express a PCCA and/or a PCCB polypeptide can be targeted to cells within a particular organ or tissue of a mammal (e.g., a human) having PA (e.g., to recapitulate the pattern of PCC expression in different tissues and/or to reduce tissue-specific damage). For example, AAVs designed to express a PCCA polypeptide and/or a PCCB polypeptide can be targeted to cells with the liver, kidney, or brain of a mammal, or can be targeted to muscle cells of a mammal. An AAV that is targeted to a particular organ or tissue can be one that infects and drives polypeptide expression at a higher level in the targeted organ or tissues than in other, non-targeted organs or tissues due to, for example, the use of a particular AAV serotype and/or selected promoter(s), or the use of a targeted injection technique such as, for example, retro-ureter injection or sub-capsular injection into the kidney, intracranial injection into the brain, intracardiac injection into the heart, or ocular injection for targeting the ocular nerve.

In some cases, the methods and materials described herein can be used to reduce or eliminate one or more symptoms or effects of PA. For example, one or more DNA virus vectors (e.g., one or more AAV vectors) designed to express a PCCA polypeptide and/or a PCCB polypeptide can be delivered to a mammal (e.g., a human) in need thereof (e.g., a human having PA) to reduce or eliminate one or more symptoms or effects of PA on a systemic or tissue-specific level. Examples of symptoms and complications of PA include, without limitation, elevated levels of propionyl carnitine (C3), methyl citrate (MeCit), and glycine, increased levels of cardiac BNP transcripts, increased cardiac or tissue triglyceride levels, increased plasma triglyceride levels, lipid droplets within cardiac or other tissues, cardiac hypertrophy, thinning of the LV wall, increased internal diameter of cardiac chambers, arrhythmia, and cardiomyopathy (e.g., dilated cardiomyopathy), which can be associated with breathlessness, swelling of the legs, ankles, and feet, abdominal bloating, fatigue, rapid, pounding or fluttering heartbeat, chest discomfort or pressure, and/or dizziness, lightheadedness and fainting. Other effects and complications of PA include, for example, hyperammonemia, leucopenia, anemia, decreased cognitive ability, growth delay, movement disorders, seizures, brain lesions, metabolic stroke, optic nerve atrophy, and acute pancreatitis. In some cases, the materials and methods described herein can be used to reduce the severity of one or more symptoms of PA in a mammal (e.g., a human) by, for example, at least 10 percent (e.g., at least 20, at least 30, at least 40, at least 50, at least 60, at least 70, at least 80, at least 90, or at least 95 percent). In some cases, the methods and materials described herein can be used to delay or prevent the development of one or more symptoms or effects of PA. For example, one or more DNA virus nucleic acids (e.g., one or more AAV vectors) designed to express a PCCA polypeptide and/or a PCCB polypeptide can be delivered to a mammal (e.g., a human) in need thereof (e.g., a human having PA) to delay or prevent the development of one or more symptoms or effects of PA.

In some cases, the methods and materials described herein can be used to reduce one or more of the following: levels of cardiac BNP transcripts, cardiac triglyceride levels, plasma triglyceride levels, accumulation or amount of lipid droplets within cardiac tissue, cardiac hypertrophy, LV wall thinning, and cardiac chamber diameter. For example, one or more DNA virus vectors (e.g., one or more AAV vectors) designed to express a PCCA polypeptide and/or a PCCB polypeptide can be delivered to a mammal (e.g., a human) in need thereof (e.g., a human having PA) to reduce cardiac BNP transcript levels by at least 10 percent (e.g., at least 20, at least 30, at least 40, at least 50, at least 60, at least 70, at least 80, at least 90, or at least 95 percent). In some cases one or more DNA virus (e.g., AAV) vectors designed to express a PCCA polypeptide and/or a PCCB polypeptide can be delivered to a mammal (e.g., a human) in need thereof (e.g., a human having PA) to reduce cardiac and/or plasma triglyceride levels by at least 10 percent (e.g., at least 20, at least 30, at least 40, at least 50, at least 60, at least 70, at least 80, at least 90, or at least 95 percent). In some cases one or more DNA virus (e.g., AAV) vectors designed to express a PCCA polypeptide and/or a PCCB polypeptide can be delivered to a mammal (e.g., a human) in need thereof (e.g., a human having PA) to reduce the accumulation or amount of lipid droplets within cardiac tissue, cardiac hypertrophy, and/or cardiac chamber diameter by at least 10 percent (e.g., at least 20, at least 30, at least 40, at least 50, at least 60, at least 70, at least 80, at least 90, or at least 95 percent). In some cases, one or more DNA virus (e.g., AAV) vectors designed to express a PCCA polypeptide and/or a PCCB polypeptide can be delivered to a mammal (e.g., a human) in need thereof (e.g., a human having PA) to increase LV wall thickness by at least 10 percent (e.g., at least 20, at least 30, at least 40, at least 50, at least 60, at least 70, at least 80, at least 90, or at least 100 percent).

In some cases, a DNA virus vector (e.g., an AAV vector) can be delivered into a patient at an early age to induce immune tolerance to the AAV vector and/or PCCA and/or PCCB proteins, which can enable repeat dosing without subsequent immune responses to the vector or the polypeptide(s) encoded by the transgene(s) in the vector.

When the methods and materials described herein are used to reduce one or more symptoms or effects of PA, the reduced symptoms or effects can be a sustained reduction. For example, one or more AAVs designed to express a PCCA polypeptide and/or a PCCB polypeptide can be delivered to a mammal (e.g., a human) in need thereof (e.g., a human having PA) to reduce levels of cardiac BNP transcripts, liver, kidney, brain, and/or cardiac triglyceride levels, plasma triglyceride levels, accumulation or amount of lipid droplets within liver, kidney, brain, and/or cardiac tissue, cardiac hypertrophy, LV wall thinning, cardiac chamber diameter, and/or brain inflammation, and/or to reduce the effects of the disease on cognition and learning in the mammal (e.g., in the germ line and/or in utero) for about from about 1 day to about 2 years (e.g., from about 1 day to about 1.5 years, from about 1 day to about 1 year, from about 1 day to about 9 months, from about 1 day to about 6 months, from about 1 day to about 3 months, from about 1 day to about 1 month, from about 1 day to about 2 weeks, from about 2 weeks to about 2 years, from about 1 month to about 2 years, from about 3 months to about 2 years, from about 6 months to about 2 years, from about 1 year to about 2 years, from about 1 week to about 1 year, from about 2 weeks to about 9 months, from about 1 month to about 6 months, from about 1 week to about 1 month, from about 1 month to about 3 months, from about 3 months to about 6 months, from about 6 months to about 9 months, from about 9 months to about 1 year, or from about 1 year to about 1.5 years).

Any appropriate DNA virus vector can be used to deliver nucleic acid encoding a PCCA polypeptide and/or nucleic acid encoding a PCCB polypeptide to a mammal. A viral vector can be derived from a positive-strand virus or a negative-strand virus. In some cases, a viral vector can be a chimeric viral vector. In some cases, a viral vector can infect dividing cells. In some cases, a viral vector can infect non-dividing cells. Examples of virus-based vectors that can be used to deliver nucleic acid encoding a PCCA polypeptide and/or a PCCB polypeptide to a mammal include, without limitation, virus-based vectors based on adenoviruses (AVs), AAVs, Herpes simplex virus (HSV), cytomegalovirus (CMV), and Epstein-Barr virus (EBV).

In some cases, when a mammal (e.g., a human) having PA is administered one or more DNA virus vectors (e.g., AAV vectors) designed to express a PCCA polypeptide and/or a PCCB polypeptide, the one or more DNA virus vectors designed to express a PCCA polypeptide and/or a PCCB polypeptide can be used to increase the efficacy of therapy and/or increase tolerance to the therapy. For example, when one or more DNA virus vectors (e.g., AAV vectors) designed to express a PCCA polypeptide and a PCCB polypeptide are delivered to a mammal (e.g., a human) in need thereof (e.g., a human having PA), co-expressing both PCCA and PCCB in the same cell can lead to balanced expression of both subunits of the enzyme to better form functional dodecamers, which can (1) increase the likelihood of producing a functional enzyme, and (2) avoid degradation of an overexpressed subunit. If only one tissue (e.g. the liver) is targeted with PCCA and or PCCB this will protect that tissue and may allow increased consumption of PCC substrates by the mammal. Increased consumption of PCC substrates may be better tolerated digestively, but these increased substrates can still be metabolized in other tissues that were not repaired, such as the heart, brain, or kidney, causing tissue-specific damage in those organs. Therefore, therapeutic delivery to PCCA and or PCCB to as many tissues as possible is likely to lead to better therapeutic outcomes. In some cases, it is noted that avoiding delivery of PCCA and or PCCB to liver can reduce the risk of liver damage and avoid insertional mutagenesis in hepatocytes and hepatocellular carcinoma.

Any appropriate mammal can be treated as described herein (e.g., by delivering one or more DNA virus vectors (e.g., AAV vectors) designed to express a PCCA polypeptide and/or a PCCB polypeptide to the mammal). Examples of mammals that can be treated as described herein include, without limitation, humans, non-human primates such as monkeys, dogs, cats, horses, cows, pigs, sheep, llamas, mice, rats, guinea pigs, and rabbits.

In some cases, a mammal (e.g., a human) can be identified as having PA using any appropriate diagnostic technique. For example, PA can be identified based on measured levels of metabolites such as propionylcarnitine and/or methylcitrate in a blood sample from a mammal, or 3-OH-propionate (3-HP) and/or methylcitrate in a urine sample from a mammal (see, e.g., Baumgartner et al. Orphanet J Rare Dis 2014, 9:130; and Grunert et al., J Inherit Metab Dis 2012, 35(1): 41-49), or by screening a mammal's DNA for pathogenic mutations in the PCCA and/or PCCB genes. In some cases, a diagnosis of PA can be made prenatally by measuring the concentration of characteristic metabolites (e.g., propionylcarnitine and/or methylcitrate) in amniotic fluid, by measuring PCC enzyme activity in a fluid or tissue sample obtained by amniocentesis or chorionic villus sampling, or molecular genetic testing.

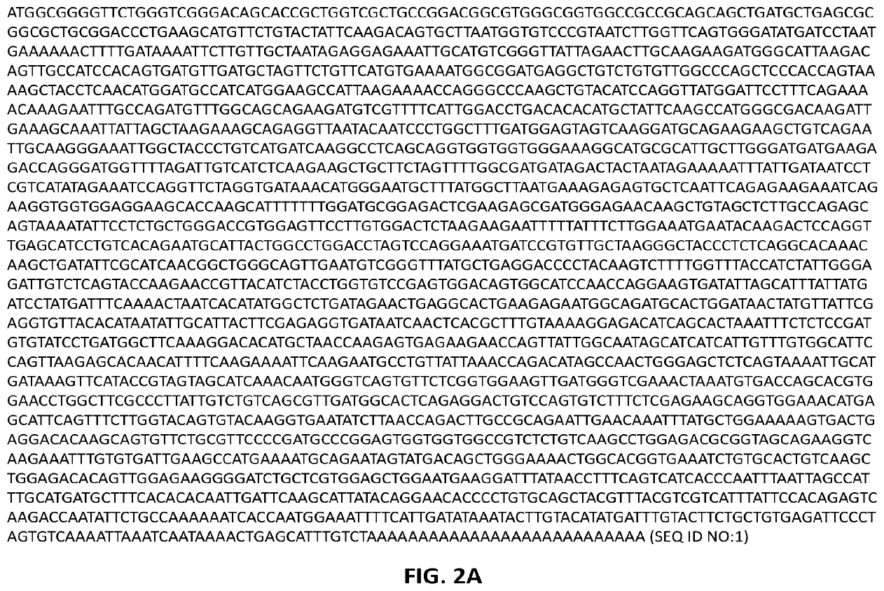

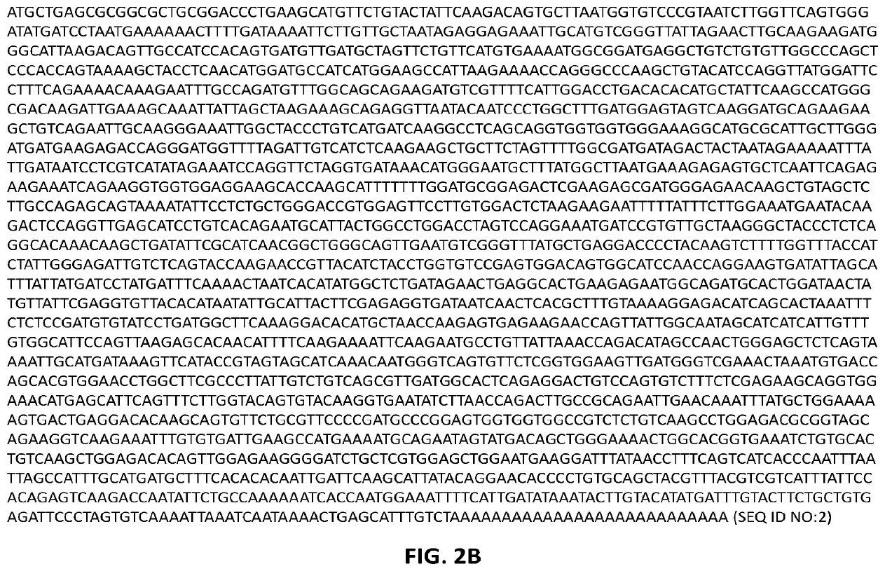

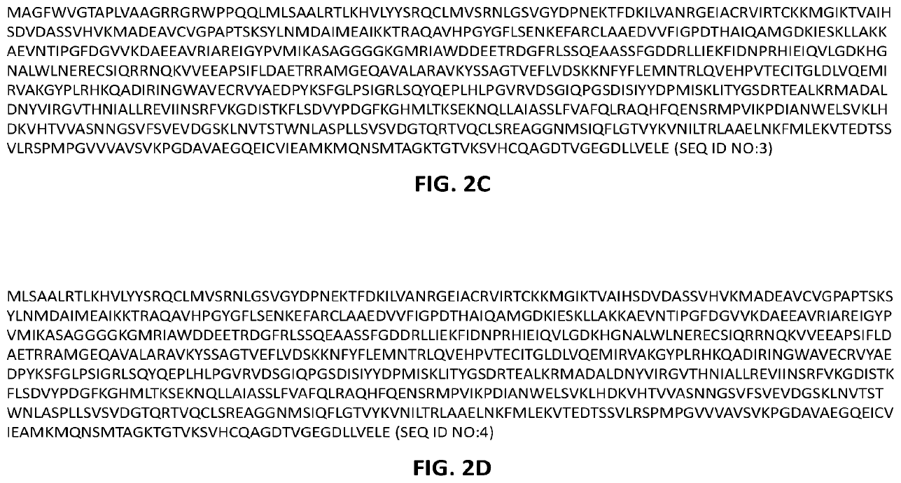

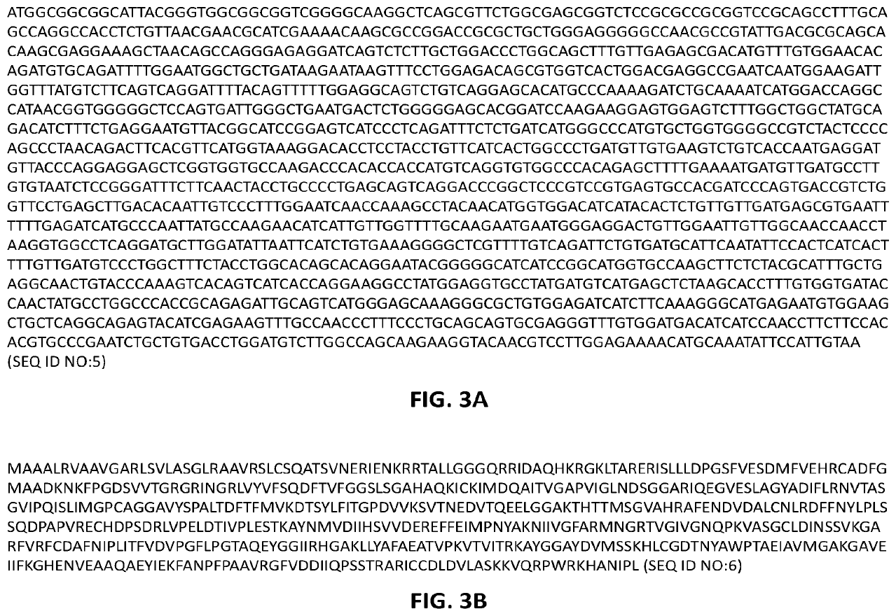

DNA virus vectors (e.g., AAV vectors) designed to express any appropriate PCCA polypeptide and/or PCCB polypeptide can be delivered to a mammal (e.g., a human) as described herein. Examples of PCA nucleic acids and PCCA polypeptides include, without limitation, those set forth in in SEQ ID NOS: 1, 2, 3, and 4 (see, FIGS. 2A, 2B, 2C, and 2D, respectively). Examples of a PCCB nucleic acid and a PCCB polypeptide include, without limitation, those set forth in SEQ ID NO:5 and SEQ ID NO:6 (see, FIGS. 3A and 3B, respectively).

In some cases, a DNA virus vector (e.g., an AAV vector) provided herein can encode a variant of a PCCA polypeptide and/or a variant of a PCCB polypeptide in place of or in addition to a nucleic acid that encodes a PCCA polypeptide and/or a PCCB polypeptide. A variant of a PCCA polypeptide can have the amino acid sequence of a naturally-occurring PCCA polypeptide with varied mitochondrial targeting sequences, and/or with one or more (e.g., e.g., one, two, three, four, five, six, seven, eight, nine, ten, or more) amino acid deletions, additions, substitutions, or combinations thereof, provided that the variant retains the function of a naturally-occurring PCCA polypeptide (e.g., catalyzing conversion of propionyl-CoA to methylmalonyl-CoA). A variant of a PCCB polypeptide can have the amino acid sequence of a naturally-occurring PCCB polypeptide with varied mitochondrial targeting sequences, and/or with one or more (e.g., e.g., one, two, three, four, five, six, seven, eight, nine, ten, or more) amino acid deletions, additions, substitutions, or combinations thereof, provided that the variant retains the function of a naturally-occurring PCCB polypeptide (e.g., catalyzing conversion of propionyl-CoA to methylmalonyl-CoA).

Any appropriate amino acid residue set forth in SEQ ID NO:3 SEQ ID NO:4, or SEQ ID NO:6 can be deleted, and any appropriate amino acid residue (e.g., any of the 20 conventional amino acid residues or any other type of amino acid such as ornithine or citrulline) can be added to or substituted within the sequence set forth in SEQ ID NO:3, SEQ ID NO:4, or SEQ ID NO:6. The majority of naturally occurring amino acids are L-amino acids, and naturally occurring polypeptides are largely comprised of L-amino acids. D-amino acids are the enantiomers of L-amino acids. In some cases, a polypeptide provided herein can contain one or more D-amino acids. In some embodiments, a polypeptide can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Amino acid substitutions can be made, in some cases, by selecting substitutions that do not differ significantly in their effect on maintaining (a) the structure of the peptide backbone in the area of the substitution, (b) the charge or hydrophobicity of the molecule at particular sites, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Non-limiting examples of substitutions that can be used herein for SEQ ID NO:3, SEQ ID NO:4, or SEQ ID NO:6 include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Further examples of conservative substitutions that can be made at any appropriate position within SEQ ID NO:3, SEQ ID NO:4, or SEQ ID NO:6 are set forth in TABLE 1 below.

In some cases, a variant of a PCCA polypeptide can be designed to include the amino acid sequence set forth in SEQ ID NO:3 or SEQ ID NO:4 with the proviso that it includes one or more non-conservative substitutions. In some cases, a variant of a PCCB polypeptide can be designed to include the amino acid sequence set forth in SEQ ID NO:6 with the proviso that it includes one or more conservative substitutions.

In some cases, a variant of a PCCA polypeptide can be designed to include the amino acid sequence set forth in SEQ ID NO:3 or SEQ ID NO:4 with the proviso that it includes one or more non-conservative substitutions. In some cases, a variant of a PCCB polypeptide can be designed to include the amino acid sequence set forth in SEQ ID NO:6 with the proviso that it includes one or more non-conservative substitutions. Non-conservative substitutions typically entail exchanging a member of one of the classes described above for a member of another class. Whether an amino acid change results in a functional polypeptide can be determined by assaying the specific activity of the polypeptide using, for example, the methods described herein.

TABLE 1
Examples of conservative amino acid substitutions

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some cases, a variant of a PCCA polypeptide having an amino acid sequence with at least 85% (e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least or 99%) sequence identity to the amino acid sequence set forth in SEQ ID NO:3 or SEQ ID NO:4, provided that it includes at least one difference (e.g., at least one amino acid addition, deletion, or substitution) with respect to SEQ ID NO:3 or SEQ ID NO:4, can be used. In some cases, a variant of a PCCB polypeptide having an amino acid sequence with at least 85% (e.g., at least 85%, at least 86%, at least 87%, at least 88%, at least 89%, at least 90%, at least 91%, at least 92%, at least 93%, at least 94%, at least 95%, at least 96%, at least 97%, at least 98%, at least or 99%) sequence identity to the amino acid sequence set forth in SEQ ID NO:6, provided that it includes at least one difference (e.g., at least one amino acid addition, deletion, or substitution) with respect to SEQ ID NO:6, can be used. Percent sequence identity is calculated by determining the number of matched positions in aligned amino acid sequences, dividing the number of matched positions by the length of the sequence set forth in the identified sequence (e.g., SEQ ID NO:1, SEQ ID NO:2, SEQ ID NO:3, SEQ ID NO:4, SEQ ID NO:5, or SEQ ID NO:6), and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number (e.g., SEQ ID NO:1, SEQ ID NO:2, SEQ ID NO:3, SEQ ID NO:4, SEQ ID NO:5, or SEQ ID NO:6) is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq -i c:\seq1.txt -j c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches by the length of the sequence set forth in the identified sequence (e.g., SEQ ID NO:1, SEQ ID NO:2, SEQ ID NO:3, SEQ ID NO:4, SEQ ID NO:5, or SEQ ID NO:6), followed by multiplying the resulting value by 100. For example, an amino acid sequence that has 680 matches when aligned with the sequence set forth in SEQ ID NO:3 is 93.4 percent identical to the sequence set forth in SEQ ID NO:3 (i.e., 680÷728×100=93.4). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Any appropriate method can be used to deliver one or more DNA virus (e.g., AAV) vectors designed to express a PCCA polypeptide and/or a PCCB polypeptide to a mammal. In some cases, DNA virus (e.g., AAV) vectors containing nucleic acid encoding a PCCA polypeptide and/or nucleic acid encoding a PCCB polypeptide can be administered to a mammal by direct injection of the DNA virus vectors. In some cases, a mammal (e.g., a human) can be administered a single administration (e.g., a single injection) of one or more DNA virus (e.g., AAV) vectors designed to express a PCCA polypeptide and/or a PCCB polypeptide. In some cases, a mammal (e.g., a human) can be administered two or more (e.g., two, three, four, or more) administrations (e.g., injections) of one or more DNA virus (e.g., AAV) vectors designed to express a PCCA polypeptide and/or a PCCB polypeptide. When a mammal is administered two or more administrations of one or more DNA virus (e.g., AAV) vectors designed to express a PCCA polypeptide and/or a PCCB polypeptide, the administrations may be no more frequent than about 30 days. In some cases, two or more administrations of one or more DNA virus (e.g., AAV) vectors designed to express a PCCA polypeptide and/or a PCCB polypeptide to a mammal can be administered at least 30 days apart, at least 180 days apart, at least 1 year apart, at least 3 years apart, at least 5 years apart, at least 8 years apart, or at least 10 years apart. For example, two or more administrations of one or more DNA virus (e.g., AAV) vectors designed to express a PCCA polypeptide and/or a PCCB polypeptide to a mammal can be administered with at least 30 days, at least 180 days, at least 1 year, at least 3 years, at least 5 years, at least 8 years, or at least 10 years apart separating the administrations. In some cases, two or more administrations of one or more DNA virus (e.g., AAV) vectors designed to express a PCCA polypeptide and/or a PCCB polypeptide to a mammal can be administered from about 30 days to about 10 years apart (e.g., from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years apart). For example, two or more administrations of one or more DNA virus vectors (e.g., one or more AAV vectors) designed to express a PCCA polypeptide and/or a PCCB polypeptide to a mammal can be administered with from about 30 days to about 10 years apart (e.g., from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years apart) separating the administrations. In some cases, two or more administrations of one or more DNA virus vectors (e.g., one or more AAV vectors) designed to express a PCCA polypeptide and/or a PCCB polypeptide to a mammal can be administered no more frequently than about 30 days. In some cases, two or more administrations of one or more DNA virus vectors (e.g., one or more AAV vectors) designed to express a PCCA polypeptide and/or a PCCB polypeptide to a mammal can be administered no more frequently than about 90 days. In some cases, two or more administrations of one or more DNA virus vectors (e.g., one or more AAV vectors) designed to express a PCCA polypeptide and/or a PCCB polypeptide to a mammal can be administered no more frequently than about 120 days. In some cases, two or more administrations of one or more DNA virus vectors (e.g., one or more AAV vectors) designed to express a PCCA polypeptide and/or a PCCB polypeptide to a mammal can be administered no more frequently than about 180 days.

DNA virus vectors (e.g., AAV vectors) encoding a PCCA polypeptide and/or a PCCB polypeptide can be delivered to a mammal for transient expression of a PCCA polypeptide and/or a PCCB polypeptide, or for stable expression of a PCCA polypeptide and/or a PCCB polypeptide. In cases where a DNA virus (e.g., an AAV) containing nucleic acid encoding a PCCA polypeptide and/or nucleic acid encoding a PCCB polypeptide is used for stable expression of a PCCA polypeptide and/or a PCCB polypeptide, the nucleic acid encoding a PCCA polypeptide and/or the nucleic acid encoding a PCCB polypeptide can be engineered to integrate into the genome of a cell. Nucleic acid can be engineered to integrate into the genome of a cell using any appropriate method. For example, gene editing techniques (e.g., CRISPR or TALEN gene editing) can be used to integrate nucleic acids designed to express a PCCA polypeptide and/or a PCCB polypeptide into the genome of a cell.

When DNA virus vectors (e.g., AAV vectors) encoding a PCCA polypeptide and/or a PCCB polypeptide are delivered to a mammal for stable expression of a PCCA polypeptide and/or a PCCB polypeptide, the expression of the PCCA polypeptide and/or a PCCB polypeptide can persist for any appropriate amount of time (e.g., following a single delivery such as a single injection). In some cases, expression of a PCCA polypeptide and/or a PCCB polypeptide can be detected within a mammal for greater than about 30 days following a single delivery (e.g., a single injection) of one or more DNA virus (e.g., AAV) vectors encoding a PCCA polypeptide and/or a PCCB polypeptide. For example, expression of a PCCA polypeptide and/or a PCCB polypeptide can be detected within a mammal for at least 30 days, at least 180 days, at least 1 year, at least 3 years, at least 5 years, at least 8 years, or at least 10 years following a single delivery (e.g., a single injection) of one or more DNA virus (e.g., AAV) vectors encoding a PCCA polypeptide and/or a PCCB polypeptide. For example, expression of a PCCA polypeptide and/or a PCCB polypeptide can be detected within a mammal for from about 30 days to about 10 years (e.g., from about 30 days to about 9 years, from about 30 days to about 8 years, from about 30 days to about 7 years, from about 30 days to about 6 years, from about 30 days to about 5 years, from about 30 days to about 4 years, from about 30 days to about 3 years, from about 30 days to about 2 years, from about 6 months to about 10 years, from about 1 year to about 10 years, from about 2 years to about 10 years, from about 3 years to about 10 years, from about 4 years to about 10 years, from about 5 years to about 10 years, from about 6 years to about 10 years, from about 7 years to about 10 years, from about 8 years to about 10 years, from about 9 years to about 10 years, from about 6 months to about 9 years, from about 1 year to about 8 years, from about 2 years to about 7 years, from about 3 years to about 6 years, from about 4 years to about 5 years, from about 1 year to about 3 years, from about 2 years to about 4 years, from about 3 years to about 5 years, from about 4 years to about 6 years, from about 5 years to about 7 years, from about 6 years to about 8 years, or from about 7 years to about 9 years) following a single delivery (e.g., a single injection) of one or more DNA virus vectors (e.g., one or more AAV vectors) encoding a PCCA polypeptide and/or a PCCB polypeptide. In some cases, stable expression of a PCCA polypeptide and/or a PCCB polypeptide can be detected within a mammal following a single injection of one or more DNA virus vectors (e.g., one or more AAV vectors) encoding a PCCA polypeptide and/or a PCCB polypeptide for about 30 days. In some cases, stable expression of a PCCA polypeptide and/or a PCCB polypeptide can be detected within a mammal following a single injection of one or more DNA virus vectors (e.g., one or more AAV vectors) encoding a PCCA polypeptide and/or a PCCB polypeptide for about 90 days. In some cases, stable expression of a PCCA polypeptide and/or a PCCB polypeptide can be detected within a mammal following a single injection of one or more DNA virus vectors (e.g., one or more AAV vectors) encoding a PCCA polypeptide and/or a PCCB polypeptide for about 120 days. In some cases, stable expression of a PCCA polypeptide and/or a PCCB polypeptide can be detected within a mammal following a single injection of one or more DNA virus vectors (e.g., one or more AAV vectors) encoding a PCCA polypeptide and/or a PCCB polypeptide for about 180 days.

When AAVs are used to deliver nucleic acid encoding PCCA polypeptide and/or nucleic acid encoding a PCCB polypeptide to a mammal as described herein, any appropriate AAV can be used. Non-limiting examples of AAVs include AAV1, AAV2, AAV3, AAV4, AAV5, AAV6, AAV7, AAV8, AAV9, AAV10, AAV11, and AAV12. In some cases, a pseudotyped AAV vector (also referred to as “mosaic” AAVs) containing a capsid from one viral serotype and a genome from another viral serotype can be used. Pseudotyped serotypes typically are denoted using a slash. For example, “AAV2/5” indicates a virus containing the genome of serotype 2 packaged in the capsid from serotype 5. In some cases, an AAV1, 8, 9, or rh10 Cap core can be synthesized with variable loops from different serotypes. For example, an AAV8 Core can be combined with AAVrh10 loops or AAV1 loops. The AAV Cap genes also can be assembled by DNA shuffling, mutagenic PCR, or propagation through mutator cells. In some cases, exogenous receptor binding or affinity tags can be inserted into the core AAV structure to target or de-target alternate cells or for purification. Assembly can occur after co-transfection of the Core and loop components.

In some cases, an AAV having a particular serotype can be used for preferential delivery to a particular tissue or organ due to the tropism of the AAV serotype. For example, an AAV1 vector can be used to target central nervous system (CNS), skeletal muscle, or heart cells, an AAV2 vector can be used to target CNS or kidney cells, an AAV4 or AAV5 vector can be used to target CNS or lung cells, an AAV6 vector can be used to target lung or skeletal muscle cells, and AAV7 vector can be used to target liver or skeletal muscle cells, an AAV8 vector can be used to target CNS, heart, liver, pancreas, or skeletal muscle cells, and an AAV9 vector can be used to target CNS, heart, liver, lung, and skeletal muscle cells. At high doses, most AAV serotypes can transduce many tissues, but have some bias. For example, AAV1 is biased toward muscle and less in liver, while AAV8 is biased toward liver and is less in muscle. In contrast, AAV9 and AAVrh10 are more broadly tropic to many tissues. In some cases, an AAV9 or AAV10 vector (e.g., an AAVrh10 vector) can be used for delivery throughout the body, as well as for crossing the blood-brain barrier for gene delivery in the brain. AAV variants generated by loop swapping, peptide insertion, shuffling, or mutation with broad tropism also can be used.

In addition to nucleic acid encoding a PCCA polypeptide and/or nucleic acid encoding a PCCB polypeptide, an AAV provided herein can contain regulatory elements operably linked to the nucleic acid encoding the PCCA polypeptide and/or PCCB polypeptide. As used herein, “operably linked” refers to positioning of a regulatory element in a vector relative to a nucleic acid in such a way as to permit or facilitate expression of the encoded RNA and/or polypeptide. Such regulatory elements can include promoter sequences, enhancer sequences, response elements, signal peptides, internal ribosome entry sequences (IRES), self-cleaving peptide sequences (e.g., P2A sequences), polyadenylation signals, terminators, or inducible elements that modulate expression (e.g., transcription or translation) of coding sequences. The choice of element(s) that may be included in an AAV depends on several factors, including, without limitation, inducibility, targeting, and the level of expression desired. For example, an enhancer/promoter combination can be included in an AAV to facilitate transcription of nucleic acid encoding a PCCA polypeptide and/or nucleic acid encoding a PCCB polypeptide. An enhancer/promoter can be a naturally occurring enhancer/promoter or a recombinant enhancer/promoter. An enhancer/promoter can be constitutive or inducible (e.g., in the presence of tetracycline or rapamycin), and can affect the expression of a nucleic acid encoding a polypeptide in a general or tissue-specific manner. Examples of enhancer/promoters that can be used to drive expression of PCCA and/or PCCB polypeptides from an AAV described herein include, without limitation, a cytomegalovirus immediate-early (CMV), Cbh, RSV, or EF-1alpha promoter for non-specific expression, a PCCA or PCCB enhancer/promoter for native expression patterns, a transthyretin promoter for liver-specific expression, a polycystin (PKD) promoter for kidney-specific expression, a synapsin promoter for neuron-specific expression, a synapsin 1, Hb9, CamkII, MeCP2, or P1e enhancer/promoter for brain-specific expression, an alpha-myosin heavy chain promoter, myosin light chain 2 promoter, or muscle creatine kinase promoter for muscle-specific expression, a cardiac troponin C promoter for heart-specific expression.

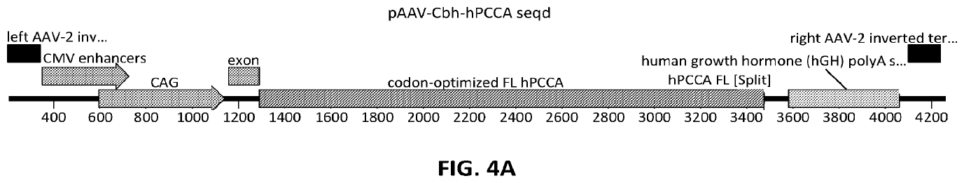

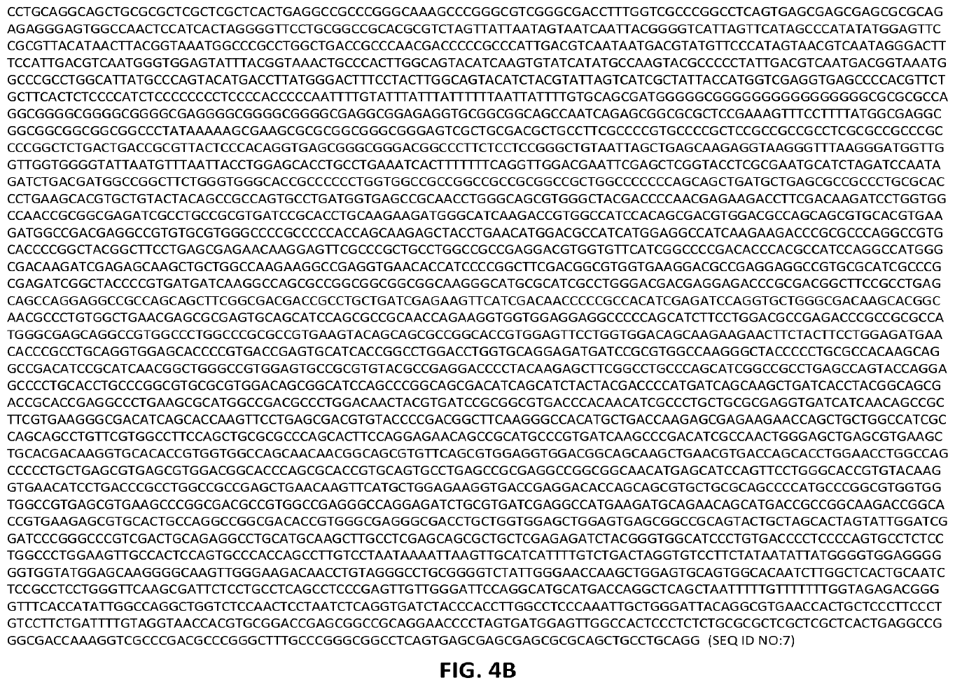

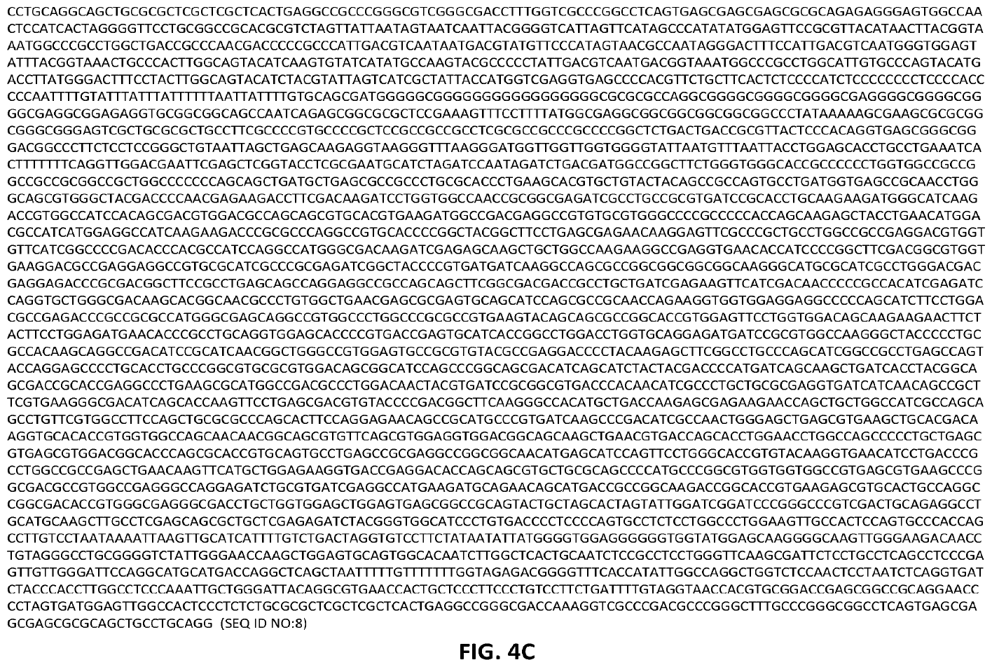

In some cases, an AAV (e.g., an AAV10) can contain a nucleotide sequence designed to express a PCCA polypeptide (e.g., a human PCCA polypeptide) operably linked to a Cbh promoter. An example of such a vector, designated “AAVrh10-Cbh-PCCA,”is illustrated in FIG. 4A, and representative nucleotide sequences for the AAVrh10-Cbh-PCCA vector are provided in FIGS. 4B and 4C (SEQ ID NOS:7 and 8).

The methods provided herein can include administering two or more AAVs designed to express a PCCA polypeptide and/or a PCCB polypeptide to a mammal (e.g., a human), in order to express PCCA and/or PCCB in different cell types. For example, a AAV1-MCK-PCCA vector designed to express PCCA polypeptides in muscle cells can be administered with an AAV8-TTR-PCCA vector designed to express PCCA polypeptides in liver cells, an AAV8-PKD-PCCA vector designed to express PCCA polypeptides in kidney cells, and/or an AAVrh10-synapsin-PCCA vector designed to express PCCA polypeptides in brain cells. In some cases, an AAV1-MCK-PCCA, AAV1-MCK-PPCB, or AAV1-MCK-PCCA-PCCB vector designed to express PCCA polypeptides and/or PCCB polypeptides in muscle cells can be administered with an AAV8-TTR-PCCA, AAV8-TTR-PCCB, or AAV8-TTR-PCCA-PCCB vector designed to express PCCA polypeptides and/or PCCB polypeptides in liver cells, an AAVrh10-Synapsin-PCCA, AAVrh10-Synapsin-PCCB, or AAVrh10-Synapsin-PCCA-PCCB vector designed to express PCCA polypeptides and/or PCCB polypeptides in neurons, and an AAV8-PKD1-PCCA, AAV8-PKD1-PCCB, or AAV8-PKD1-PCCA-PCCB vector designed to express PCCA polypeptides and/or PCCB polypeptides in kidney cells. In some cases, an AAVrh10-MCK-PCCA, AAVrh10-MCK-PCCB, or AAVrh10-MCK-PCCA-PCCB vector designed to express PCCA polypeptides and/or PCCB polypeptides in muscle cells can be administered with an AAVrh10-TTR-PCCA, AAVrh10-TTR-PCCB, or AAVrh10-TTR-PCCA-PCCB vector designed to express PCCA polypeptides and/or PCCB polypeptides in liver cells, an AAVrh10-Synapsin-PCCA, AAVrh10-Synapsin-PCCB, or AAVrh10-Synapsin-PCCA-PCCB vector designed to express PCCA polypeptides and/or PCCB polypeptides in neurons, and an AAVrh10-PKD1-PCCA, AAVrh10-PKD1-PCCB, or AAVrh10-PKD1-PCCA-PCCB vector designed to express PCCA polypeptides and/or PCCB polypeptides in kidney cells. In some cases, an AAVrh10-MCK-PCCA, AAVrh10-MCK-PCCB, or AAVrh10-MCK-PCCA-PCCB vector designed to express PCCA polypeptides and/or PCCB polypeptides in muscle cells can be administered with an AAVrh10-TTR-PCCA, AAVrh10-TTR-PCCB, or AAVrh10-TTR-PCCA-PCCB vector designed to express PCCA polypeptides and/or PCCB polypeptides in liver cells, an AAVrh10-Synapsin-PCCA, AAVrh10-Synapsin-PCCB, or AAVrh10-Synapsin-PCCA-PCCB vector designed to express PCCA polypeptides and/or PCCB polypeptides in neurons, and an AAV8-PKD1-PCCA, AAV8-PKD1-PCCB, or AAV8-PKD1-PCCA-PCCB vector designed to express PCCA polypeptides and/or PCCB polypeptides in kidney cells.

The AAVs to be combined can all be designed to express PCCA polypeptides, or the AAVs can all be designed to express PCCB polypeptides, or some of the AAVs in the combination can be designed to express PCCA polypeptides and other AAVs in the combination can be designed to expression PCCB polypeptides.

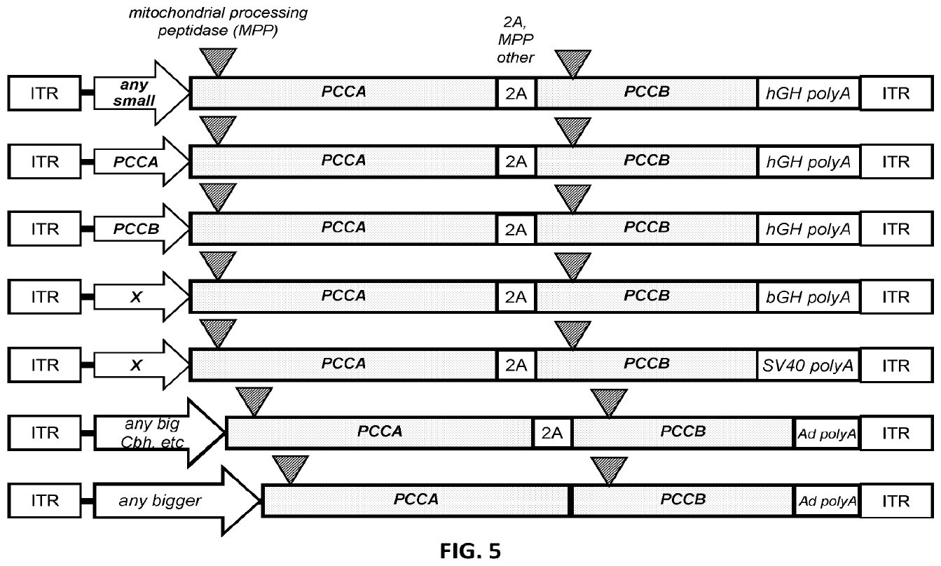

In some cases, AAVs can be designed to contain nucleic acid that encodes both a PCCA polypeptide and a PCCB polypeptide. Examples of such AAVs are illustrated in FIG. 5. The nucleotide sequence encoding the PCCA polypeptide can be positioned upstream from the nucleotide sequence encoding the PCCB polypeptide, as shown in FIG. 5, or the order can be reversed such that the nucleotide sequence encoding the PCCB polypeptide is upstream from the nucleotide sequence encoding the PCCA polypeptide. The AAV also can include other elements, such as inverted terminal repeats (ITRs), one or more promoters, a polyA sequence, one or more self-cleaving peptide (e.g., P2A) sequences, and one or more mitochondrial peptidase processing (MPP) sites. For example, the sequences encoding PCCA and PCCB can be operably linked to separate promoters, or the sequences encoding PCCA and PCCB can be controlled by a single promoter and separated by a self-cleaving peptide sequence.

In some cases, a nucleic acid that encodes PCCA and PCCB can be designed to minimize the size of the cDNA encoding PCCA and PCCB. For example, a nucleic acid can include only one mitochondrial targeting sequence, a minimized synthetic or natural mitochondrial targeting sequence, a deletion of N- and/or C-terminal portions of PCCA and/or PCCB that are not necessary for subunit assembly and function, or a reduction in the size of amino acid chains that are not involved in direct subunit to subunit contacts. Modifications to reduce the overall size of an AAV or other vector expression cassette can include, for example, use of a single ITR or packaging element rather than two, a truncated or internally-deleted ITR or packaging element, a minimized enhancer/promoter sequence, a minimized polyA signal for mammalian poly-adenylation of AATAAA and expansions thereon, a direct poly A sequence (e.g. AAAAAAAA), and nuclease cleavage sites flanking the PCCA and PCCB sequence, with or without homology regions to enable insertion into the nuclear or mitochondrial genome.

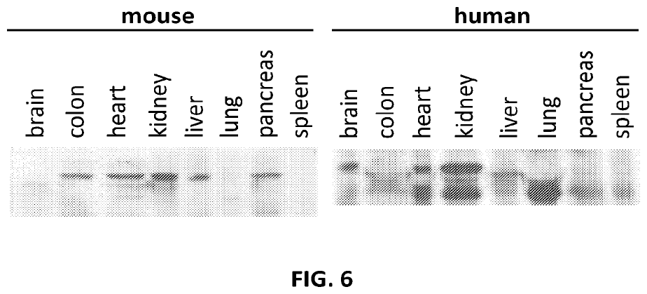

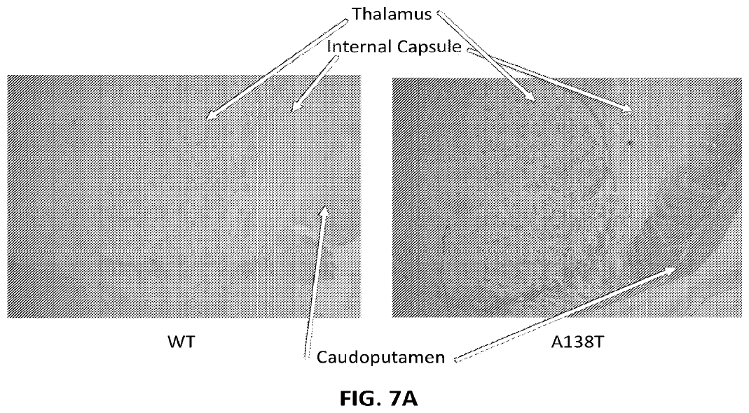

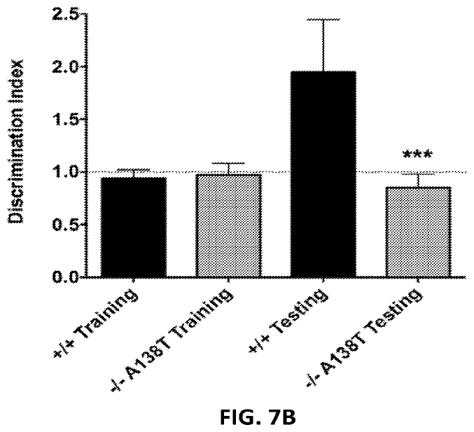

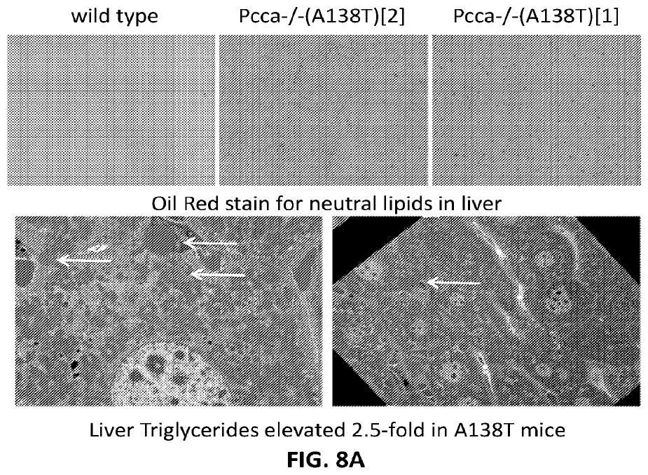

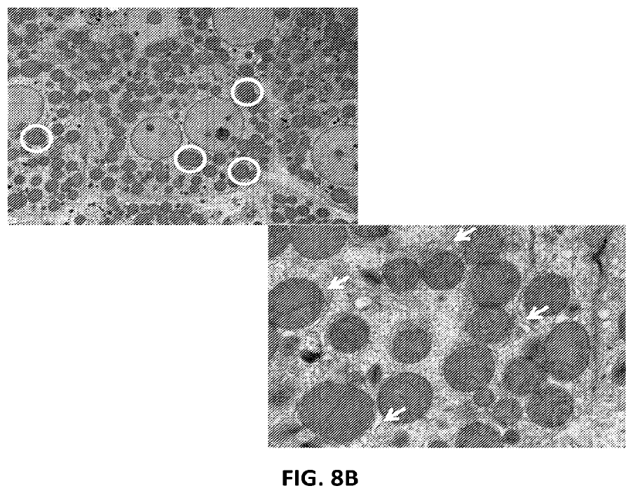

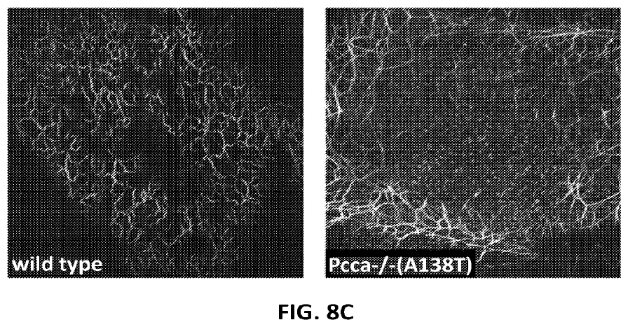

Most potential gene therapies for PA are aimed at the liver. However, western blotting for PCCA in human and mouse tissues revealed that the highest expression of PCCA occurs in the kidney (FIG. 6), and Northern blotting indicated that the same is true for PCCB. Likewise, damage due to PA manifests in many tissues beyond the liver, including the brain, kidney, heart, nerves, and muscles. For example, as indicated by studies using A138T mice, PA can result in increased brain inflammation (FIG. 7A) and can adversely affect cognition and learning (FIG. 7B). In the liver, PA can increase lipids and triglycerides (FIGS. 8A and 8C), and can alter liver structures related to mitochondria and the ER (FIG. 8B). Thus, in some cases, this document provides AAVs designed to express a PCCA polypeptide and/or a PCCB polypeptide in the kidney and other sites. For example, an AAVrh10 vector designed to express a PCCA polypeptide and/or a PCCB polypeptide, in combination (e.g., simultaneously or sequentially) with an AAV1 vector or another viral vector designed to express a PCCA polypeptide and/or a PCCB polypeptide, can be administered to a mammal (e.g., a human) having PA to provide broad gene delivery to tissues and the brain via AAVrh10 while enhancing delivery to cardiac and skeletal muscle with AAV1. Such as strategy also can be employed to reduce gene delivery to the liver relative to AAV8 gene therapy to reduce the risk of liver damage and hepatocellular carcinoma. This can be further restricted by use of enhancers/promoters or miRNA target sequences that attenuate expression in the liver. Conversely, combination of AAVrh10 with AAV8 can provide broad gene delivery to tissues and the brain via AAVrh10 while enhancing delivery to the liver and kidney with AAV8. Combining AAVrh10 with AAV8 with AAV can provide broad gene delivery to tissues and the brain via AAVrh10 while enhancing delivery to the liver and kidney with AAV8 and enhancing delivery to muscles with AAV1.

In some cases, gene therapy can provoke new or recall T cell responses against AAV capsid or neo-antigens in PCCA or PCCB proteins that were not present in the mutant proteins. In such cases, modified (repaired) cells can be targeted by T cell responses and eliminated. To address this potential issue, AAVs designed to express a PCCA polypeptide and/or a PCCB polypeptide can include a sequence encoding proteins that can compete for binding of ubiquitinated proteins to the proteasome such as ubiquitin, poly-ubiquitin, or a Rad23 polypeptide. Rad23 is an adaptor protein that binds to both ubiquitinated substrates and to the proteasome, but escapes degradation because it lacks an effective initiation region at which the proteasome could engage the Rad23 protein and unfold it. Co-expression of Rad23 can protect proteins from proteasome degradation as well as small molecule proteasome inhibitors. Thus, Rad23 co-expression with AAV or PCCA or PCCB can reduce degradation of these proteins, thus reducing display of their peptides on MHC I to reduce detection and elimination of the cells by T cells. The inclusion of a Rad23 coding sequence in the constructs provided herein can prolong the effectiveness of the PCCA and PCCB polypeptides that also are encoded by the constructs. The Rad23 polypeptide can be a full length Rad23 polypeptide, or can be a fragment of a Rad23 polypeptide.

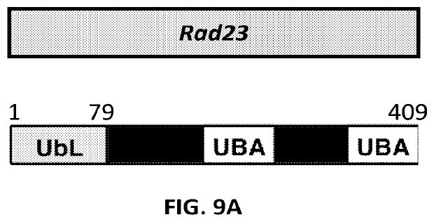

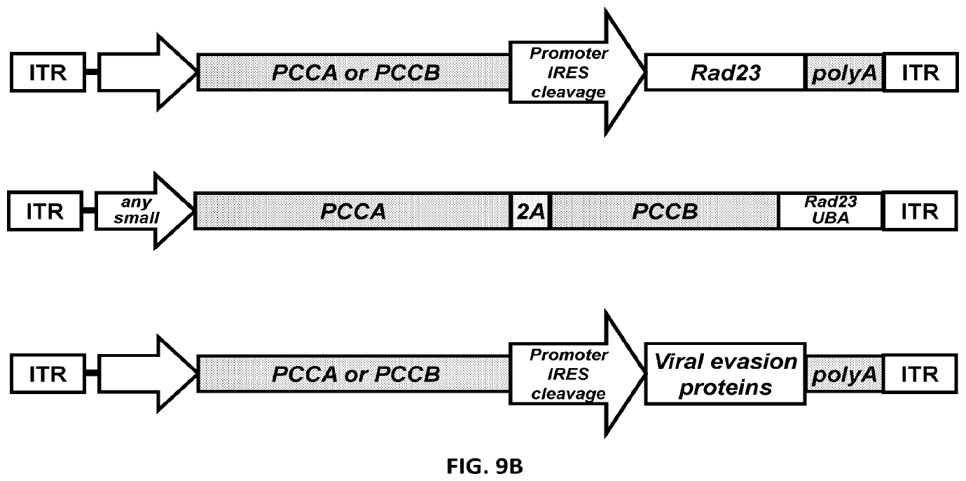

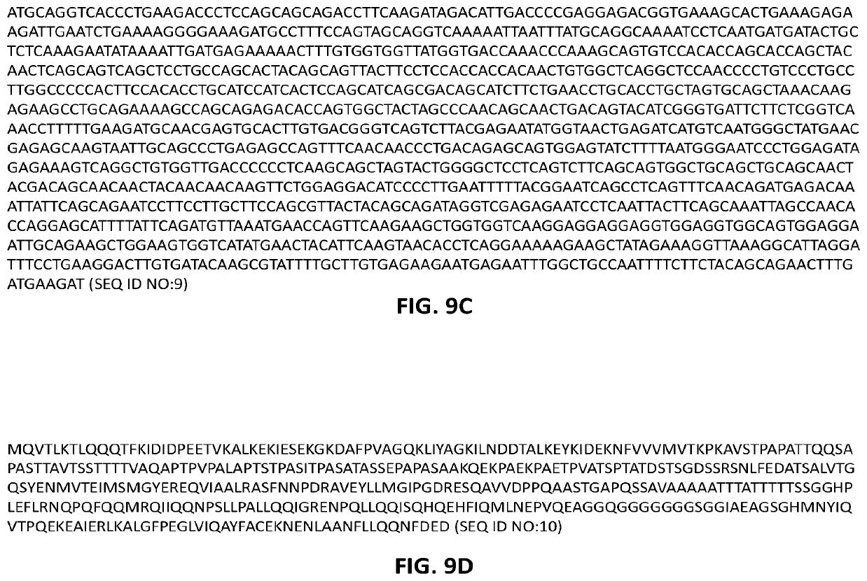

FIG. 9A shows the general structure of Rad23, indicating the positions of the UbL and UBA domains. FIG. 9B depicts the structures of several AA Vs designed to express a PCCA polypeptide and/or a PCCB polypeptide with a Rad23 polypeptide, a UBA domain from a Rad23 polypeptide, or another viral evasion polypeptide. Exemplary Rad23 nucleotide and amino acid sequences (SEQ ID NOS:9 and 10, respectively) are provided in FIGS. 9C and 9D.

As used herein, the term “AAV particle” refers to packaged capsid forms of the AAV virus that transmits its nucleic acid genome to cells. In some cases, a composition containing an AAV particle encoded by an AAV vector as provided herein can be administered at a concentration from about 10¹⁰ AAV particles/mL to about 10¹⁵ AAV particles/mL (e.g., from about 10¹⁰ AAV particles/mL to about 10¹¹ AAV particles/mL, from about 10¹⁰ AAV particles/mL to about 10¹² AAV particles/mL, from about 10¹⁰ AAV particles/mL to about 10¹³ AAV particles/mL, from about 10¹¹ AAV particles/mL to about 10¹² AAV particles/mL, from about 10¹¹ AAV particles/mL to about 10¹³ AAV particles/mL, from about 10¹¹ AAV particles/mL to about 10¹⁴ AAV particles/mL, from about 10¹² AAV particles/mL to about 10¹³ AAV particles/mL, from about 10¹² AAV particles/mL to about 10¹⁴ AAV particles/mL, or from about 10¹³ AAV particles/mL to about 10¹⁴ AAV particles/mL).

In some cases, AAVs encoding a PCCA polypeptide and/or a PCCB polypeptide can be administered to a mammal by direct injection, or by administering one or more the AAVs as complexes with lipids, polymers, or nanospheres. In some cases, AAVs designed to express a PCCA polypeptide and/or a PCCB polypeptide can be delivered to a mammal (e.g., a human) via direct injection (e.g., into a particular organ or tissue), intraperitoneal administration, intranasal administration, intravenous administration, intrathecal administration, intracerebral administration, intraparenchymal administration, or oral delivery.

In some cases, AAVs encoding a PCCA polypeptide and/or a PCCB polypeptide also can contain nucleic acid encoding a detectable label. For example, an AAV provided herein can include nucleic acid encoding a PCCA polypeptide and/or nucleic acid encoding a PCCB polypeptide, and nucleic acid encoding a detectable label positioned such that the encoded PCCA and/or PCCB polypeptide is a fusion polypeptide that includes a PCCA polypeptide and/or a PCCB polypeptide fused to a detectable polypeptide. In some cases, a detectable label can be a peptide tag. Examples of detectable labels that can be used as described herein include, without limitation, an HA tag, a Myc-tag, a FLAG-tag, and a fluorescent polypeptide (e.g., a green fluorescent polypeptide (GFP)).

Nucleic acid encoding a PCCA polypeptide and/or nucleic acid encoding a PCCB polypeptide can be produced by techniques including, without limitation, common molecular cloning, polymerase chain reaction (PCR), chemical nucleic acid synthesis techniques, and combinations of such techniques. For example, PCR or RT-PCR can be used with oligonucleotide primers designed to amplify nucleic acid (e.g., genomic DNA or RNA) encoding a PCCA polypeptide and/or a PCCB polypeptide.

In some cases, one or more AAVs designed to express a PCCA polypeptide and/or a PCCB polypeptide can be formulated into a composition (e.g., a pharmaceutical composition) for administration to a mammal (e.g., a human). For example, one or more AAVs designed to express a PCCA polypeptide and/or a PCCB polypeptide can be formulated into a pharmaceutically acceptable composition for administration to a mammal (e.g., a human) having PA. In some cases, one or more AAVs designed to express a PCCA polypeptide and/or a PCCB polypeptide can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin.

A composition (e.g., a pharmaceutical composition) containing one or more AAVs designed to express a PCCA polypeptide and/or a PCCB polypeptide can be formulated into any appropriate dosage form. Examples of dosage forms include solid or liquid forms including, without limitation, gels, liquids, suspensions, solutions (e.g., sterile solutions), sustained-release formulations, and delayed-release formulations.

A composition (e.g., a pharmaceutical composition) containing one or more AAVs designed to express a PCCA polypeptide and/or a PCCB polypeptide can be designed for parenteral (e.g., intravenous, intraperiotoneal, intramuscular, or intrathecal) administration. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

A composition (e.g., a pharmaceutical composition) containing one or more AAVs designed to express a PCCA polypeptide and/or a PCCB polypeptide can be administered locally or systemically. For example, a composition containing one or more AAVs designed to express a PCCA polypeptide and/or a PCCB polypeptide can be administered locally by injection into a particular tissue or organ of a mammal (e.g., a human). For example, a composition containing one or more AAVs designed to express a PCCA polypeptide and/or a PCCB polypeptide can be administered locally by intracardiac injection to the heart of a mammal (e.g., a human).

An effective amount (e.g., effective dose) of one or more AAVs designed to express a PCCA polypeptide and/or a PCCB polypeptide can vary depending on the severity of disease, the route of administration, the age and general health condition of the subject, excipient usage, the possibility of co-usage with other therapeutic treatments such as use of other agents, and/or the judgment of the treating physician.

An effective amount of a composition (e.g., a pharmaceutical composition) containing one or more AAVs designed to express a PCCA polypeptide and/or a PCCB polypeptide can be any amount that can treat the mammal without producing significant toxicity to the mammal. An effective amount of one or more AAVs designed to express a PCCA polypeptide and/or a PCCB polypeptide can be any appropriate amount. For example, an effective amount of AAVs designed to express a PCCA polypeptide and/or a PCCB polypeptide can be from about 1×10¹¹ viral genomes per kg (vg/kg) per dose to about 4×10¹⁴ vg/kg per dose (e.g., from about 1×10¹¹ vg/kg to about 1×10¹² vg/kg, from about 1×10¹² vg/kg to about 1×10¹³ vg/kg, from about 1×10¹³ vg/kg to about 1×10¹⁴ vg/kg, from about 1×10¹¹ vg/kg to about 1×10¹³ vg/kg, or from about 1×10¹² vg/kg to about 1×10¹⁴ vg/kg per dose). In some cases, an effective amount of AAVs designed to express a PCCA polypeptide and/or a PCCB polypeptide can be from about 1×10¹² vg/kg to about 1×10¹³ vg/kg (e.g., about 3.28×10¹² vg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the PA in the mammal may require an increase or decrease in the actual effective amount administered.

The frequency of administration of a composition (e.g., a pharmaceutical composition) containing one or more AAVs designed to express a PCCA polypeptide and/or a PCCB polypeptide can be any frequency that can treat the PA without producing significant toxicity to the mammal. For example, the frequency of administration can be from about once a week to about once a month, from about once every two week to once every other month, or from about once a month to about once a year. The frequency of administration can remain constant or can be variable during the duration of treatment. A course of treatment with a composition containing AAVs designed to express a PCCA polypeptide and/or a PCCB polypeptide provided herein can include rest periods. For example, a composition containing one or more AAVs designed to express a PCCA polypeptide and/or a PCCB polypeptide can be administered daily over a six-week period. As with the effective amount, various factors can influence the actual frequency of administration used for a particular application. For example, the effective amount, duration of treatment, use of multiple treatment agents, route of administration, and severity of the PA in the mammal may require an increase or decrease in administration frequency.

An effective duration for administering a composition (e.g., a pharmaceutical composition) containing one or more AAVs designed to express a PCCA polypeptide and/or a PCCB polypeptide can be any duration that treat the PA without producing significant toxicity to the mammal. For example, the effective duration can vary from several weeks to several months or years. In some cases, the effective duration for the treatment of PA can range in duration from about one month to about a lifetime. Multiple factors can influence the actual effective duration used for a particular treatment. For example, an effective duration can vary with the frequency of administration, effective amount, use of multiple treatment agents, route of administration, and severity of the PA in the mammal.

In some cases, the one or more AAVs designed to express a PCCA polypeptide and/or a PCCB polypeptide can be used as the sole active agent used to treat a mammal having PA. In some cases, the methods and materials described herein can include subjecting a mammal having PA to one or more (e.g., one, two, three, four, five or more) additional treatments (e.g., therapeutic interventions) that are effective to treat PA. Examples of additional treatments that can be used as described herein to treat PA include, without limitation, a protein managed diet, and use of medications such as carnitine. In some cases, the one or more additional treatments that are effective to treat PA can be performed at the same time as the administration of the one or more AAVs designed to express a PCCA polypeptide and/or a PCCB polypeptide. In some cases, the one or more additional treatments that are effective to treat PA can be performed before and/or after the administration of the one or more AAVs designed to express a PCCA polypeptide and/or a PCCB polypeptide.

The invention will be further described in the following examples, which do not limit the scope of the invention described in the claims.

### EXAMPLES

### Example 1—Materials and Methods

Animal Studies: All institutional and national guidelines for the care and use of laboratory animals were followed. All animals were housed and bred in the Department of Comparative Medicine animal facilities (Mayo Clinic, Rochester, MN). Pcca−/− (A138T) mice are on an FVB genetic background and were generated and characterized as described elsewhere (Guenzel et al., Molecular Therapy: The Journal of the American Society of Gene Therapy 2013, 21(7): 1316-1323). In particular, this mouse model of PA was generated by rescuing the lethal phenotype of Pcca gene knockout in mice by transgenesis with a human hypomorphic PCCA transgene harboring an A138T mutation. A138T mice have 2% of wild-type liver PCC activity and recapitulate many of the biochemical aspects of PA disease, including systemic elevations in propionylcarnitine, methylcitrate, glycine, and ammonia. A138T mice also appear to manifest phenotypes suggestive of clinical manifestations observed in PA patients, including increased cardiac mass and elevations in cardiac mRNA transcript levels for BNP (Guenzel et al, supra) a marker of cardiac dysfunction (Wallen et al., Heart 1997, 77(3):264-267; Yasue et al., Circulation 1994, 90(1): 195-203; and Maisel, Circulation 2002, 105(20):2328-2331).

Metabolic Assays: Cardiac tissue was removed from mice euthanized by exsanguination. After thoroughly rinsing the heart tissue in cold PBS, 20 mg was removed and homogenized in water using a glass cell homogenizer. After sonicating and centrifuging to remove debris, acylcarnitines in the lysate were measured by tandem mass spectrometry using methods described elsewhere (Cox et al., Human Mol Genet 2001, 10(19):2069-2077; and Smith et al., Curr Protoc Hum Genet 2010, Chapter 17:Unit 17 18, 11-20). Propionylcarnitine and methylcitrate also were measured by tandem mass spectrometry in blood collected via submandibular puncture with GOLDENROD™ lancets (MEDIpoint Inc., Mineola, NY) and spotted on WHATMANR 903 Protein Saver filter paper cards (GE Healthcare, Westborough, MA). Punches from the card were then analyzed by tandem mass spectrometry as described elsewhere (Turgeon et al., Clinical Chem 2008, 54(4):657-664; and Turgeon et al., Clinical Chem 2010, 56(11):1686-1695).

Triglyceride Quantification: Tissue samples were removed upon euthanasia by exsanguination and rinsed in PBS. Ten (10) to 20 mg were then homogenized in 0.05% TWEEN® solution using a mini-beadbeater (Biospec, Bartlesville, OK) on “homogenize” setting. Samples were then incubated at 70° C. for 5 minutes and centrifuged for 3 minutes at 2500×g. Absorbance at 540 nm was recorded before plasma and tissue lysate samples along with Thermo Trace Triglyceride standards were combined with Infinity Triglyceride Reagent (Thermo Fischer Scientific, Rockford, IL) and incubated for 30 minutes at 37° C. Results from tissue samples were normalized to total protein content determined by BCA assay (Thermo Fischer Scientific).

Mitochondrial Sizing. Images of cardiac tissue obtained by transmission electron microscopy at 5000-fold magnification were loaded into ImageJ software (imagej.nih.gov/ij/). Outline traces of >100 mitochondria were made from each of 3 images for both wild type and Pcca−/− (A138T) mice.

Echocardiography: A skilled sonographer who was blinded to experimental conditions performed echocardiograms on mice anesthetized with isoflurane using a GE Vivid 7 Dimension echocardiography system along with an I13L probe (GE Healthcare, Westborough, MA) at 12 MHz. Short axis LV images were used to calculate parameters, including thickness of the LV posterior wall (LVPW) and intraventricular septum (IVS), left ventricular (LV) mass, and left ventricular inner diameter (LVID).

Transmission Electron Microscopy: Tissues were collected immediately after mice were euthanized by exsanguination and placed in Trump's fixative (1% glutaraldehyde/4% formalin in 0.1 M phosphate buffer), post-fixed in 1% osmium tetroxide, dehydrated in ethanol, and embedded in Spurr epoxy resin. The tissue was sectioned at 800 angstroms, placed onto copper grids and stained with lead citrate. Sections were viewed using a model 1400 transmission electron microscope (JEOL, Tokyo, Japan) at an accelerating voltage of 80 KeV.

AAV Vector Production and Administration: All AAV vectors were produced by triple transfection of HEK-293 cells with pHelper, pR/C 2/1 or 2/8, and pss-MCK6-hPCCA or pss-TTR-hPCCA. AAV was purified from culture media using tangential flow filtration followed by iodixanol ultracentrifugation, as described elsewhere, (Guenzel et al., supra; and Guenzel et al., Human Gene Ther 2015, 26(3): 153-160). AAV vectors were administered intravenously, intraperitoneally, by retroureter, by sub-capsular, by intracranial, and by oral delivery.

PCC Enzyme Activity Assay: PCC activity levels were determined as described elsewhere (Jiang et al., J Biol Chem 2005, 280(30):27719-27727). Briefly, mouse hearts were homogenized in lysis buffer (50 mM Tris pH 8.0, 1 mM DTT, 1 mM EDTA, protease inhibitor cocktail) and centrifuged for 30 minutes at 15,000 rpm in a benchtop centrifuge. 75 μg of protein was used as determined by the Bradford method for radiometric determination of PCC activity.

Cardiac BNP Transcript Assay: BNP transcript levels were quantitated as described elsewhere (Peche et al., Cell Mol Life Sci: CMLS 2013, 70(3):527-543) using BNP primers and GAPDH reference primers as described therein. SYBR® Green Master Mix (Life Technologies, Grand Island, NY) was used in 384 well plates, and data collection and analysis occurred on a VIIA™ 7 real-time PCR system with associated software (Life Technologies, Grand Island, NY).

### Example 2—Pcca^{-/-}(A138T) Mice Accumulate High Levels of Intracardiac and Systemic Propionylcarnitine and Lipids

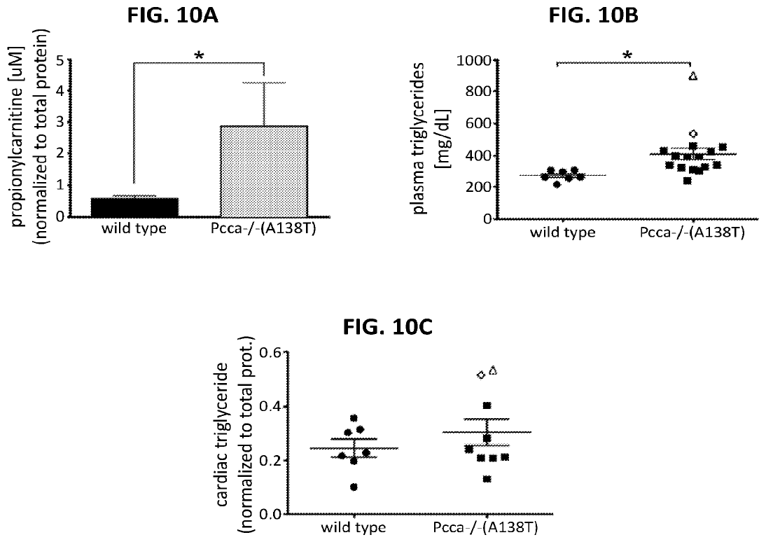

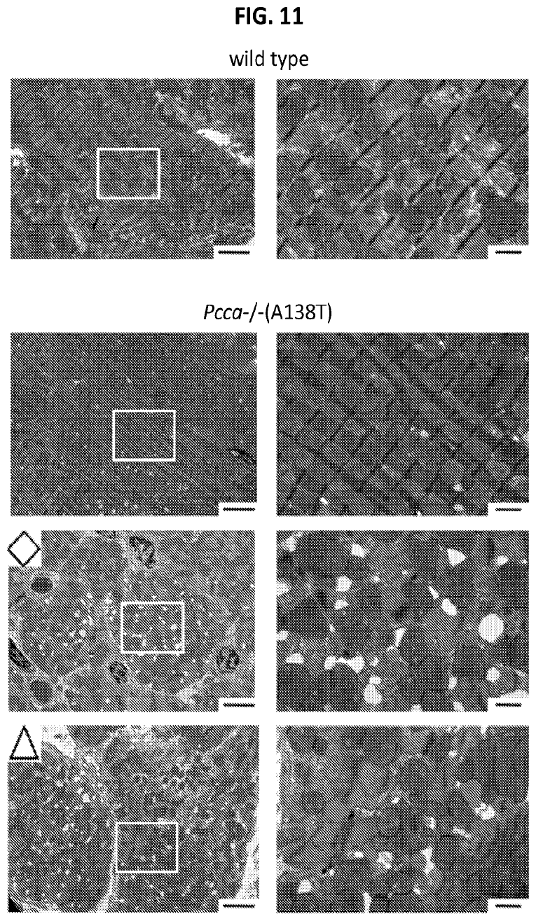

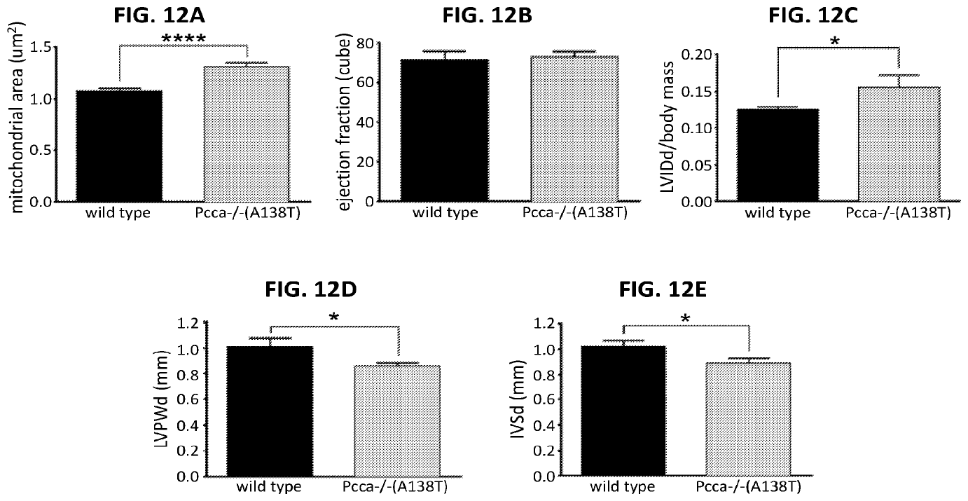

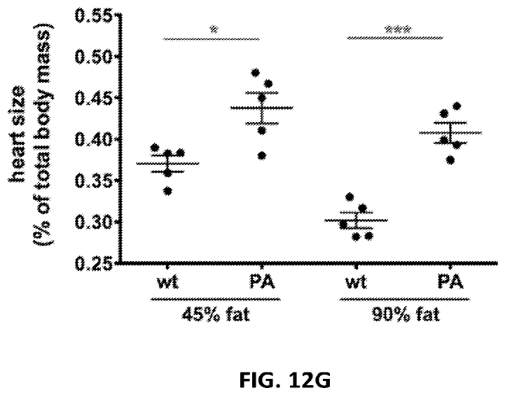

Elevations in blood propionylcarnitine and other metabolites have been documented in A138T mice (Guenzel et al. 2013, supra; and Guenzel et al. 2015, supra). To determine whether similar elevations were present in the hearts of the A138T mice, propionylcarnitine levels were measured in cardiac tissue lysates from normal and PA mice. These assays revealed that propionylcarnitine levels were four-fold higher in the A138T mice than in control animals (p<0.05, FIG. 10A). Mitochondrial dysfunction inherent to PA can lead to lipid accumulation in tissue, and lipid droplets have been detected in muscle tissue from PA patients (Schwab et al., Biochem J 2006, 398(1): 107-112). Cardiac tissues from the mice were analyzed by transmission electron microscopy (TEM). Hearts from wild type mice contained regular sarcomere banding patterns with mitochondria evenly dispersed in relatively straight bands, with no lipid droplets (FIG. 11, top row). In contrast, there were increased numbers of light grey and white circular bodies consistent with the presence of lipid droplets in hearts from A138T mice. To examine this further, triglyceride (TG) levels were measured in cardiac tissues and in plasma taken from wild type and PA mice. Plasma TG levels were elevated in A138T mice relative to wild type animals (p<0.05, FIG. 10B), but cardiac TG levels for all of these animals were not statistically different due to wide variations in individual levels (FIG. 10C). Two of the nine mice that were sampled had markedly elevated TG levels in their plasma and cardiac tissues (denoted as open symbols in FIGS. 10B and 10C). When the hearts from these two mice were analyzed by TEM, markedly higher numbers of lipid droplets were observed (FIG. 11, bottom two rows compared to top two rows), similar to abnormal lipid findings observed in PA patients (Schwab et al., supra). In addition, TEMs of the cardiac tissues from the two mice with elevated myocardial TGs revealed highly disordered sarcomere and mitochondrial banding patterns (FIG. 11, bottom two rows compared to top two rows). As tissue lipid accumulation also can be attributed to mitochondrial dysfunction, mitochondrial phenotypes in hearts of the PA mice also were assessed. The average mitochondrial area was larger in A138T vs. normal mice (p<0.0001, FIG. 12A).

### Example 3—Cardiac Structure and Function in Pcca^{-/-}(A138T) Mice

As reported elsewhere, cardiac/body mass ratio was increased in A138T mice sacrificed at 8 months (Guenzel et al. 2013; supra). Since both dilated and hypertrophic cardiomyopathies have been reported in PA patients (Massoud and Leonard, Eur J Pediatr 1993, 152(5):441-445; Laemmle et al., Eur J Pediatr 2014, 173(7):971-974; Romano et al., J Pediatr 2010, 156(1): 128-134; and Mardach et al., Mol Genet Metab 2005, 85(4):286-290), echocardiography was performed in 12-week-old mice to better characterize the cardiac phenotype. Ejection fractions were similar in A138T and wild type mice (FIG. 12B). Indexed left ventricular internal diameter end diastole (LVIDd) (FIG. 12C) tended to be higher in A138T mice, whereas thickness of the LV posterior wall diastole (LVPWd) and intraventricular septum diastole (IVSd) were lower (FIGS. 12D and 12E), suggesting the presence of an early dilated rather than hypertrophic cardiomyopathy phenotype in this PA mouse model. In addition, heart mass was elevated in A138T mice as compared to wild type mice, whether the animals were fed a low fat diet or a high fat diet (FIG. 12F).

### Example 4—Gene Therapy Corrects Cardiac PCC Deficiency in Pcca^{-/-}(A138T) Mice of Both Sexes

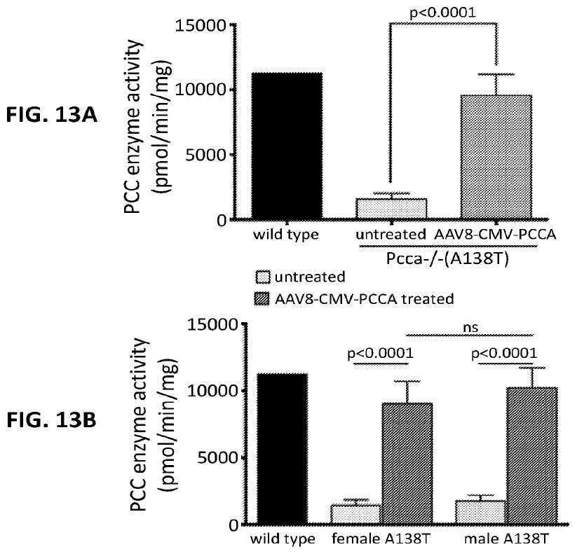

Gene therapy can correct genetic defects in any cell that a therapeutic vector is able to transduce, including the liver and many other tissues. Gene delivery by the AAV8 serotype is most efficient in liver hepatocytes, but this vector also delivers genes to many other tissues, including the heart (Wallen et al., supra; Yasue et al., supra; Maisel et al., supra; and Guenzel et al. 2015, supra). To determine if systemic therapy with AAV8 affects cardiac PCC function, A138T mice were treated with 5×10¹¹ viral genomes (vg) of AAV serotype 8 expressing human PCCA under CMV control (AAV8-CMV-hPCCA). Treated and control A138T mice were allowed to age 1.5 years and then were sacrificed to measure PCC activity in their hearts. Old, 1.5 year old A138T mice had about 14% of wild type PCC enzyme activity (TABLE 2 and FIG. 13A). In contrast, PCC activity in the hearts of AAV8-CMV-hPCCA treated animals was increased to 80% of wild type levels in female mice and to 90% of wild type levels in male mice (p<0.0001 compared to sex-matched untreated A138T, TABLE 2 and FIG. 13B). These data suggested that systemic AAV gene therapy not only reduces systemic metabolites associated with the disease, but also repairs function in tissues such as the heart long after a single treatment.

TABLE 2
Cardiac PCC enzyme activity

| | untreated | | | AAV8-CMV-hPCCA | |
|---|---|---|---|---|---|
| | wild type | female | male | female | male |
| | (1) | (8) | (6) | (5) | (4)* |
| cardiac PCC | 11246 | 1451 ± | 1746 ± | 9029 ± | 10232 ± |
| activity | | 407 | 452 | 1675 | 1479 |
| (pmol/min/mg) | | | | | |

*p < 0.05

### Example 5—Muscle-Targeted PCCA Gene Therapy Corrects Cardiac Abnormalities, but Liver-Targeted Therapy does not

As discussed above, AAV8-CMV-hPCCA led to repaired function in the liver, the heart, and other tissues. Liver- and muscle-targeted AAV vectors were then examined for PCCA gene therapy. To engineer these vectors, the natural tissue transduction biases of AAV8 for the liver and AAV serotype 1 (AAV1) for muscle were made more specific by the use of tissue specific promoters (Guenzel et al. 2015, supra). In particular, AAV8 was made more liver specific by placing PCCA under the control of the liver-specific transthyretin promoter (AAV8-TTR-PCCA), and AAV1 was made more muscle specific by placing PCCA under the control of the muscle creatine kinase promoter (AAV1-MCK6-PCCA). After intravenous injection, AAV8-TTR-PCCA mediated expression only in the liver of A138T mice, while AAV1-MCK-PCCA produced expression only in cardiac and skeletal muscle (Guenzel et al. 2015, supra). In addition, both targeted vectors mediated significant reductions in PCC metabolites in the blood. This suggested that genetic repair of the either tissue would reduce their production of PA metabolites and/or that they can both serve to absorb systemic metabolites. These results did not, however, address whether these targeted therapies mediated any functional repairs in the targeted tissues.

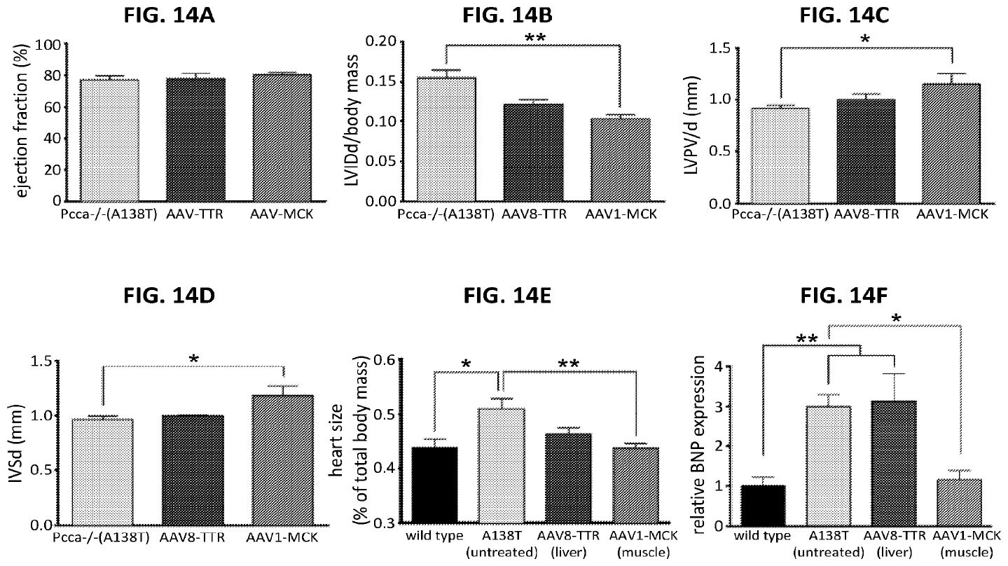

To determine the effects of the targeted vectors on the cardiac phenotypes of PA, A138T mice were injected intravenously with 5×10¹¹ viral genomes (vg) of AAV8-TTR-PCCA or AAV1-MCK-PCCA, and cardiac parameters were compared with untreated A138T mice 8 months later. Echocardiograms revealed no differences in ejection fraction between groups (FIG. 14A), consistent with a lack of this phenotype in the A138T mice (FIG. 12B). Indexed LVIDd was decreased significantly in muscle targeted AAV1-MCK-PCCA treated mice when compared to A138T mice (p<0.01, FIG. 14B). LVPWd and IVSd wall thicknesses also were increased significantly in muscle targeted AAV1-MCK-PCCA treated mice when compared to A138T mice 230 (p<0.05, FIGS. 14C and 14D). In contrast, wall thicknesses were not significantly improved in mice treated with liver targeted AAV8-TTR-PCCA (FIGS. 14B-14D). At autopsy, the cardiac-to-body mass ratio in AAV1-MCK-PCCA treated mice was reduced significantly when compared to untreated A138T mice (p<0.01, FIG. 14E). This ratio was reduced in AAV8-TTR-PCCA treated mice, but did not reach statistical significance. A138T mice had elevations in BNP transcript levels in their hearts when compared to control mice (FIG. 14F), consistent with studies described elsewhere (Guenzel et al. 2013, supra). Treatment of the mice with liver-specific AAV8-TTR-PCCA had no beneficial effect on BNP transcript levels in the heart. In contrast, muscle-targeted gene therapy with AAV1-MCK-PCCA reduced BNP transcripts significantly relative to untreated mice (p<0.001, FIG. 14F). BNP levels in AAV1-MCK-PCCA treated mice were indistinguishable from those in wild type mice (FIG. 14F).

Taken together, the results disclosed herein establish and characterize a natural cardiac phenotype in Pcca^{-/-} (A138T) mice that is likely an authentic recapitulation of cardiomyopathy in people with PA. These results indicate that cardiomyopathy in PA is correctable with muscle-specific gene therapy to deliver the deficient PCC subunit.

### Example 6—Treating Mice with Two Gene Therapy Vectors to Express PCCA and PCCB Simultaneously

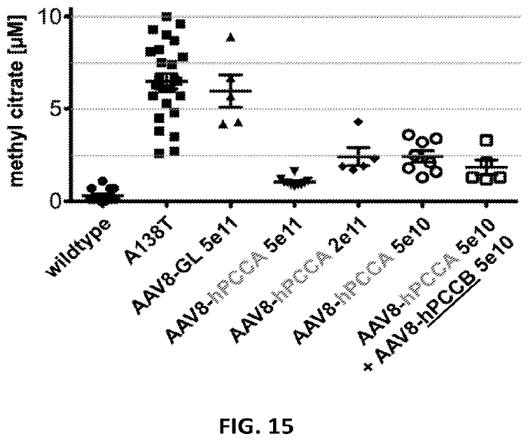

Mice were administered a combination of vectors expressing PCCA and PCCB, or were administered a vector expressing only PCCA. Combined intravenous delivery of both AAV8-PCCA and AAV8-PCCB mediated greater reductions in methyl citrate (MeCit) than delivery of AAV8-PCCA alone in A138T mice with mutations in only PCCA (FIG. 15). A138T PCCA mutant mice treated with 5×10¹⁰ vg of AAV8-PCCA plus 5×10¹⁰ vg of AAV8-PCCB had lower methyl citrate levels than mice treated with an equal amount of 5×10¹⁰ vg of AAV8-PCCA or with a higher amount of 1×10¹¹ vg of AAV8-PCCA. Methyl citrate levels of A138T PCCA mutant mice treated with 5×10¹⁰ vg of AAV8-PCCA plus 5×10¹⁰ vg of AAV8-PCCB approached the levels of mice that were treated with ten times higher levels of AAV8-PCCA alone (5×10¹¹ vg) (FIG. 15). The increased efficacy may have been due to equal expression of both of subunits of the PCC dodecamer rather than imbalanced expression of only one subunit.

### Example 7—Treating Mice with a Single Vector Expressing PCCA and PCCB

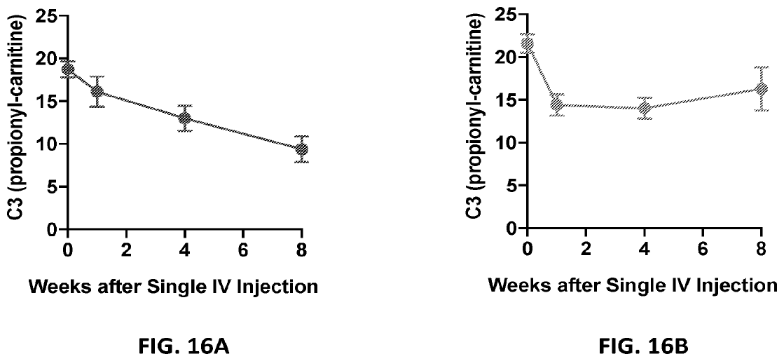

Pcca^{-/-}(A138T) mice were administered 1×10¹² vg of AAVrh10-RSV-PCCA-P2A-PCCB that carries a fusion protein of human PCCA with human PCCB. Mice were injected intravenously and propionylcarnitine (C3) levels were measured in blood over the course of 8 weeks. In male animals, C3 levels continued to decline throughout the course of the study (FIG. 16A). In female animals, however, the level of C3 was rapidly reduced after vector administration, but then plateaued and eventually began to rise as the study progressed (FIG. 16B). These data indicated that the PCCA-P2A-PCCB transgene was able to mediate therapeutic reductions in PA metabolites after intravenous AAV therapy. The efficacy may have been due to equal expression of both of subunits of the PCC dodecamer rather than imbalanced expression of only one subunit.

### Example 8—Comparison of Muscle-Specific Expression from Broad Tropism AAVrh10 and Muscle-Biased AAV1

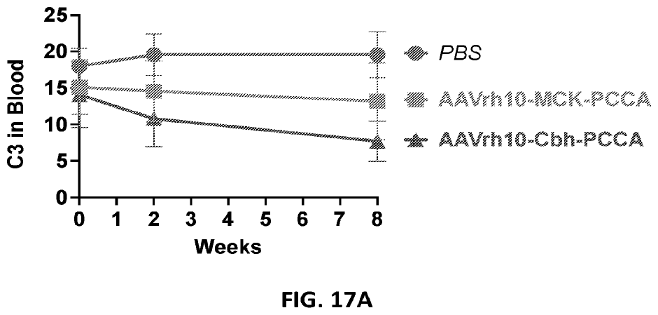

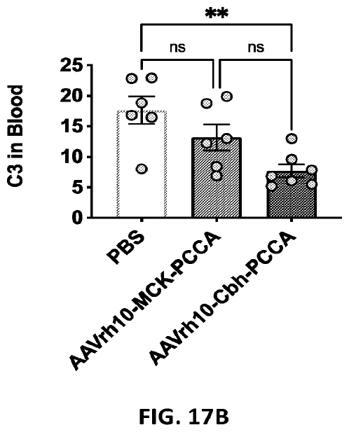

Studies were conducted to test the effect of treatment with a broad tropism vector vs. a muscle-biased vector on blood propionyl carnitine levels in vivo. Pcca−/−A138T mice were injected intravenously with PBS or 5×10¹¹ viral genomes of broad tropism AAVrh10 with muscle-specific MCK promoter driving PCCA expression, or muscle-biased AAV1 with muscle-specific MCK promoter driving PCCA expression, and blood propionyl carnitine (C3) levels were measured at 0, 2, and 8 weeks after treatment. FIG. 17A is a graph plotting C3 levels over time, while FIG. 17B is a graph plotting C3 levels on week 8. These studies demonstrated that the broad expression of the construct led to a greater decrease in blood C3 levels (as compared to PBS control) than muscle-specific expression.

### Example 9—Comparison of Various Configurations of Expression Construct Components

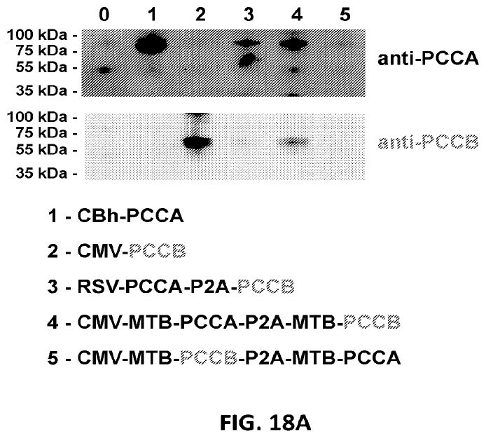

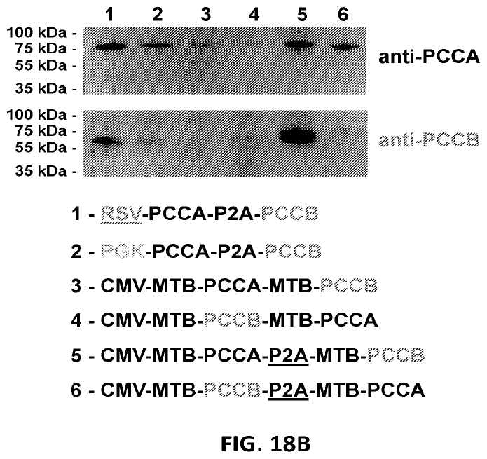

Additional studies were carried out to evaluate expression from constructs containing several different configurations of the components in the expression plasmid. In particular, PCCA and PCCB single or fusion proteins were expressed from coding sequences in different arrangements within expression constructs, with and without modified mitochondrial targeting sequences and P2A elements. 293-G1A-PCCA-knock-out cells were transfected with the plasmids indicated in FIGS. 18A and 18B using lipofectamine plus. After 24 hours, the cells were harvested and mitochondria were prepared using a Milteny kit. Mitochondrial extracts were analyzed by western blot with monoclonal antibodies against PCCA (1:2000) or PCCB (1:5000) followed by goat anti-mouse IgG-HRP (1:10000). Blots were imaged using West Femto Chemiluminescent reagent (Pierce). FIG. 18A shows a western blot comparing expression from single codon-optimized PCCA and PCCB expression plasmids with Cbh or CMV promotors to PCCA-PCCB or PCCB-PCCA fusion protein expression vectors with RSV or CMV promoters. FIG. 18B shows a western blot comparing expression from different PCCA-PCCB and PCCB-PCCA fusion protein vectors with and without native or wobbled (MTB) mitochondrial targeting sequences and with or without a P2A element before the second MTB targeting sequence. Also shown is expression from RSV, PGKi, and CMV promoters. These studies demonstrated that only a subset of PCCA-PCCB fusion proteins could generate the proteins to allow both to be targeted into mitochondria. These data also showed that cellular and viral enhancer/promoters (e.g. PGK, RSV, CMV, Cbh) could be used to drive PCCA and PCCB expression.

### Example 10—Additional Vectors

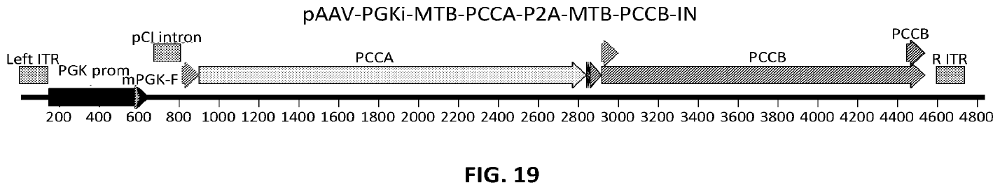

Additional vectors are designed that enable expression of PCCA and/or PCCB in target tissues. The additional vectors are based, for example, on the AAV vector depicted in FIG. 19, such that they are small enough to be packaged into an AAV particle and can express both PCCA and PCCB.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An adeno-associated virus serotype rh10 (AAVrh10) nucleic acid construct comprising:
a nucleotide sequence encoding a propionyl-CoA carboxylase A (PCCA) polypeptide or a propionyl-CoA carboxylase B (PCCB) polypeptide, and
a Cbh promoter operably linked to said nucleotide sequence encoding said PCCA polypeptide or said PCCB polypeptide.

2. The AAVrh10 nucleic acid construct of claim 1, wherein said nucleotide sequence encoding said PCCA polypeptide or said PCCB polypeptide is codon optimized for expression in humans.

3. The AAVrh10 nucleic acid construct of claim 1, wherein said nucleotide sequence encodes a PCCA polypeptide comprising the sequence set forth in SEQ ID NO:3 or SEQ ID NO:4, or a sequence at least 95% identical to SEQ ID NO:3 or SEQ ID NO:4.

4. The AAVrh10 nucleic acid construct of claim 1, wherein said nucleotide sequence encodes a PCCB polypeptide comprising the sequence set forth in SEQ ID NO:6, or a sequence at least 95% identical to SEQ ID NO:6.

5. The AAVrh10 nucleic acid construct of claim 1, wherein said construct further comprises a nucleotide sequence encoding a Rad23 polypeptide or a portion thereof.

6. An AAV nucleic acid construct comprising:
a first nucleotide sequence encoding a PCCA polypeptide,
a second nucleotide sequence encoding a PCCB polypeptide, and
a promoter operably linked to said first nucleotide sequence encoding said PCCA polypeptide.

7. The AAV nucleic acid construct of claim 6, wherein said first nucleotide sequence encoding said PCCA polypeptide and said second nucleotide sequence encoding said PCCB polypeptide are codon optimized for expression in humans.

8. The AAV nucleic acid construct of claim 6, wherein said first nucleotide sequence encodes a PCCA polypeptide comprising the sequence set forth in SEQ ID NO:3 or SEQ ID NO:4, or a sequence at least 95% identical to SEQ ID NO:3 or SEQ ID NO:4, and wherein said second nucleotide sequence encodes a PCCB polypeptide comprising the sequence set forth in SEQ ID NO:6, or a sequence at least 95% identical to SEQ ID NO:6.

9. The AAV nucleic acid construct of claim 6, wherein said AAV is AAV1, AAV8, AAV9, or AAVrh10.

10. The AAV nucleic acid construct of claim 6, wherein said construct further comprises a nucleotide sequence encoding a Rad23 polypeptide or a portion thereof.

11. A composition comprising:
(a) a first AAV nucleic acid construct comprising a first promoter operably linked to a nucleotide sequence encoding a PCCA polypeptide or a PCCB polypeptide, wherein said first AAV nucleic acid construct is an AAVrh10 nucleic acid construct, and
(b) a second AAV nucleic acid comprising a first promoter operably linked to a nucleotide sequence encoding a PCCA polypeptide or a PCCB polypeptide, wherein said second AAV nucleic acid construct is not an AAVrh10 nucleic acid construct.

12. The composition of claim 11, wherein said second AAV nucleic acid construct is an AAV1 nucleic acid construct or an AAV8 nucleic acid construct.

13. The composition of claim 11, wherein said first promoter is a Cbh promoter, said second promoter is a Cbh promoter, or both said first promoter and said second promoter are Cbh promoters.

14. The composition of claim 11, wherein said first or second AAV nucleic acid construct comprises a nucleotide sequence encoding a PCCA polypeptide, and wherein said PCCA polypeptide comprises the sequence set forth in SEQ ID NO:3 or SEQ ID NO:4, or a sequence at least 95% identical to SEQ ID NO:3 or SEQ ID NO:4.

15. The composition of claim 11, wherein said first or second AAV nucleic acid construct comprises a nucleotide sequence encoding a PCCB polypeptide, and wherein said PCCB polypeptide comprises the sequence set forth in SEQ ID NO:6, or a sequence at least 95% identical to SEQ ID NO:6.

16. The composition of claim 11, wherein said first AAV nucleic acid further comprises a nucleotide sequence encoding a Rad23 polypeptide or a portion thereof, said second AAV nucleic acid further comprises a nucleotide sequence encoding a Rad23 polypeptide or a portion thereof, or both said first AAV nucleic acid and said second AAV nucleic acid further comprises a nucleotide sequence encoding a Rad23 polypeptide or a portion thereof.

17. A method for treating a mammal having propionic acidemia (PA), wherein said method comprises administering to said mammal the AAVrh10 nucleic acid construct of claim 1, wherein said administering results in an increased level of a PCCA polypeptide and/or a PCCB polypeptide in said mammal.

18. A method for treating a mammal having PA, wherein said method comprises administering to said mammal the AAV nucleic acid construct of claim 6, wherein said administering results in an increased level of a PCCA polypeptide and/or a PCCB polypeptide in said mammal.

19. A method for treating a mammal having PA, wherein said method comprises administering to said mammal the composition of claim 11, wherein said administering results in an increased level of a PCCA polypeptide and/or a PCCB polypeptide in said mammal.


SEQUENCE LISTING (10 sequences)

SEQ ID NO: 1 - DNA, 2480 bp, Homo sapiens
atggcggggttctgggtcgggacagcaccgctggtcgctgccggacggcgtgggcggtggccgccgcagcagctgatgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatgaaggatttataacctttcagtcatcacccaatttaattagccatttgcatgatgctttcacacacaattgattcaagcattatacaggaacacccctgtgcagctacgtttacgtcgtcatttattccacagagtcaagaccaatattctgccaaaaaatcaccaatggaaattttcattgatataaatacttgtacatatgatttgtacttctgctgtgagattccctagtgtcaaaattaaatcaataaaactgagcatttgtctaaaaaaaaaaaaaaaaaaaaaaaaaaa

SEQ ID NO: 2 - DNA, 2405 bp, Homo sapiens
atgctgagcgcggcgctgcggaccctgaagcatgttctgtactattcaagacagtgcttaatggtgtcccgtaatcttggttcagtgggatatgatcctaatgaaaaaacttttgataaaattcttgttgctaatagaggagaaattgcatgtcgggttattagaacttgcaagaagatgggcattaagacagttgccatccacagtgatgttgatgctagttctgttcatgtgaaaatggcggatgaggctgtctgtgttggcccagctcccaccagtaaaagctacctcaacatggatgccatcatggaagccattaagaaaaccagggcccaagctgtacatccaggttatggattcctttcagaaaacaaagaatttgccagatgtttggcagcagaagatgtcgttttcattggacctgacacacatgctattcaagccatgggcgacaagattgaaagcaaattattagctaagaaagcagaggttaatacaatccctggctttgatggagtagtcaaggatgcagaagaagctgtcagaattgcaagggaaattggctaccctgtcatgatcaaggcctcagcaggtggtggtgggaaaggcatgcgcattgcttgggatgatgaagagaccagggatggttttagattgtcatctcaagaagctgcttctagttttggcgatgatagactactaatagaaaaatttattgataatcctcgtcatatagaaatccaggttctaggtgataaacatgggaatgctttatggcttaatgaaagagagtgctcaattcagagaagaaatcagaaggtggtggaggaagcaccaagcatttttttggatgcggagactcgaagagcgatgggagaacaagctgtagctcttgccagagcagtaaaatattcctctgctgggaccgtggagttccttgtggactctaagaagaatttttatttcttggaaatgaatacaagactccaggttgagcatcctgtcacagaatgcattactggcctggacctagtccaggaaatgatccgtgttgctaagggctaccctctcaggcacaaacaagctgatattcgcatcaacggctgggcagttgaatgtcgggtttatgctgaggacccctacaagtcttttggtttaccatctattgggagattgtctcagtaccaagaaccgttacatctacctggtgtccgagtggacagtggcatccaaccaggaagtgatattagcatttattatgatcctatgatttcaaaactaatcacatatggctctgatagaactgaggcactgaagagaatggcagatgcactggataactatgttattcgaggtgttacacataatattgcattacttcgagaggtgataatcaactcacgctttgtaaaaggagacatcagcactaaatttctctccgatgtgtatcctgatggcttcaaaggacacatgctaaccaagagtgagaagaaccagttattggcaatagcatcatcattgtttgtggcattccagttaagagcacaacattttcaagaaaattcaagaatgcctgttattaaaccagacatagccaactgggagctctcagtaaaattgcatgataaagttcataccgtagtagcatcaaacaatgggtcagtgttctcggtggaagttgatgggtcgaaactaaatgtgaccagcacgtggaacctggcttcgcccttattgtctgtcagcgttgatggcactcagaggactgtccagtgtctttctcgagaagcaggtggaaacatgagcattcagtttcttggtacagtgtacaaggtgaatatcttaaccagacttgccgcagaattgaacaaatttatgctggaaaaagtgactgaggacacaagcagtgttctgcgttccccgatgcccggagtggtggtggccgtctctgtcaagcctggagacgcggtagcagaaggtcaagaaatttgtgtgattgaagccatgaaaatgcagaatagtatgacagctgggaaaactggcacggtgaaatctgtgcactgtcaagctggagacacagttggagaaggggatctgctcgtggagctggaatgaaggatttataacctttcagtcatcacccaatttaattagccatttgcatgatgctttcacacacaattgattcaagcattatacaggaacacccctgtgcagctacgtttacgtcgtcatttattccacagagtcaagaccaatattctgccaaaaaatcaccaatggaaattttcattgatataaatacttgtacatatgatttgtacttctgctgtgagattccctagtgtcaaaattaaatcaataaaactgagcatttgtctaaaaaaaaaaaaaaaaaaaaaaaaaaa

SEQ ID NO: 3 - protein, 728 aa, Homo sapiens
MAGFWVGTAPLVAAGRRGRWPPQQLMLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 4 - protein, 703 aa, Homo sapiens
MLSAALRTLKHVLYYSRQCLMVSRNLGSVGYDPNEKTFDKILVANRGEIACRVIRTCKKMGIKTVAIHSDVDASSVHVKMADEAVCVGPAPTSKSYLNMDAIMEAIKKTRAQAVHPGYGFLSENKEFARCLAAEDVVFIGPDTHAIQAMGDKIESKLLAKKAEVNTIPGFDGVVKDAEEAVRIAREIGYPVMIKASAGGGGKGMRIAWDDEETRDGFRLSSQEAASSFGDDRLLIEKFIDNPRHIEIQVLGDKHGNALWLNERECSIQRRNQKVVEEAPSIFLDAETRRAMGEQAVALARAVKYSSAGTVEFLVDSKKNFYFLEMNTRLQVEHPVTECITGLDLVQEMIRVAKGYPLRHKQADIRINGWAVECRVYAEDPYKSFGLPSIGRLSQYQEPLHLPGVRVDSGIQPGSDISIYYDPMISKLITYGSDRTEALKRMADALDNYVIRGVTHNIALLREVIINSRFVKGDISTKFLSDVYPDGFKGHMLTKSEKNQLLAIASSLFVAFQLRAQHFQENSRMPVIKPDIANWELSVKLHDKVHTVVASNNGSVFSVEVDGSKLNVTSTWNLASPLLSVSVDGTQRTVQCLSREAGGNMSIQFLGTVYKVNILTRLAAELNKFMLEKVTEDTSSVLRSPMPGVVVAVSVKPGDAVAEGQEICVIEAMKMQNSMTAGKTGTVKSVHCQAGDTVGEGDLLVELE

SEQ ID NO: 5 - DNA, 1620 bp, Homo sapiens
atggcggcggcattacgggtggcggcggtcggggcaaggctcagcgttctggcgagcggtctccgcgccgcggtccgcagcctttgcagccaggccacctctgttaacgaacgcatcgaaaacaagcgccggaccgcgctgctgggagggggccaacgccgtattgacgcgcagcacaagcgaggaaagctaacagccagggagaggatcagtctcttgctggaccctggcagctttgttgagagcgacatgtttgtggaacacagatgtgcagattttggaatggctgctgataagaataagtttcctggagacagcgtggtcactggacgaggccgaatcaatggaagattggtttatgtcttcagtcaggattttacagtttttggaggcagtctgtcaggagcacatgcccaaaagatctgcaaaatcatggaccaggccataacggtgggggctccagtgattgggctgaatgactctgggggagcacggatccaagaaggagtggagtctttggctggctatgcagacatctttctgaggaatgttacggcatccggagtcatccctcagatttctctgatcatgggcccatgtgctggtggggccgtctactccccagccctaacagacttcacgttcatggtaaaggacacctcctacctgttcatcactggccctgatgttgtgaagtctgtcaccaatgaggatgttacccaggaggagctcggtggtgccaagacccacaccaccatgtcaggtgtggcccacagagcttttgaaaatgatgttgatgccttgtgtaatctccgggatttcttcaactacctgcccctgagcagtcaggacccggctcccgtccgtgagtgccacgatcccagtgaccgtctggttcctgagcttgacacaattgtccctttggaatcaaccaaagcctacaacatggtggacatcatacactctgttgttgatgagcgtgaattttttgagatcatgcccaattatgccaagaacatcattgttggttttgcaagaatgaatgggaggactgttggaattgttggcaaccaacctaaggtggcctcaggatgcttggatattaattcatctgtgaaaggggctcgttttgtcagattctgtgatgcattcaatattccactcatcacttttgttgatgtccctggctttctacctggcacagcacaggaatacgggggcatcatccggcatggtgccaagcttctctacgcatttgctgaggcaactgtacccaaagtcacagtcatcaccaggaaggcctatggaggtgcctatgatgtcatgagctctaagcacctttgtggtgataccaactatgcctggcccaccgcagagattgcagtcatgggagcaaagggcgctgtggagatcatcttcaaagggcatgagaatgtggaagctgctcaggcagagtacatcgagaagtttgccaaccctttccctgcagcagtgcgagggtttgtggatgacatcatccaaccttcttccacacgtgcccgaatctgctgtgacctggatgtcttggccagcaagaaggtacaacgtccttggagaaaacatgcaaatattccattgtaa

SEQ ID NO: 6 - protein, 539 aa, Homo sapiens
MAAALRVAAVGARLSVLASGLRAAVRSLCSQATSVNERIENKRRTALLGGGQRRIDAQHKRGKLTARERISLLLDPGSFVESDMFVEHRCADFGMAADKNKFPGDSVVTGRGRINGRLVYVFSQDFTVFGGSLSGAHAQKICKIMDQAITVGAPVIGLNDSGGARIQEGVESLAGYADIFLRNVTASGVIPQISLIMGPCAGGAVYSPALTDFTFMVKDTSYLFITGPDVVKSVTNEDVTQEELGGAKTHTTMSGVAHRAFENDVDALCNLRDFFNYLPLSSQDPAPVRECHDPSDRLVPELDTIVPLESTKAYNMVDIIHSVVDEREFFEIMPNYAKNIIVGFARMNGRTVGIVGNQPKVASGCLDINSSVKGARFVRFCDAFNIPLITFVDVPGFLPGTAQEYGGIIRHGAKLLYAFAEATVPKVTVITRKAYGGAYDVMSSKHLCGDTNYAWPTAEIAVMGAKGAVEIIFKGHENVEAAQAEYIEKFANPFPAAVRGFVDDIIQPSSTRARICCDLDVLASKKVQRPWRKHANIPL

SEQ ID NO: 7 - DNA, 4036 bp, Artificial
  synthetic oligonucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgtcaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggacgaattcgagctcggtacctcgcgaatgcatctagatccaatagatctgacgatggccggcttctgggtgggcaccgcccccctggtggccgccggccgccgcggccgctggcccccccagcagctgatgctgagcgccgccctgcgcaccctgaagcacgtgctgtactacagccgccagtgcctgatggtgagccgcaacctgggcagcgtgggctacgaccccaacgagaagaccttcgacaagatcctggtggccaaccgcggcgagatcgcctgccgcgtgatccgcacctgcaagaagatgggcatcaagaccgtggccatccacagcgacgtggacgccagcagcgtgcacgtgaagatggccgacgaggccgtgtgcgtgggccccgcccccaccagcaagagctacctgaacatggacgccatcatggaggccatcaagaagacccgcgcccaggccgtgcaccccggctacggcttcctgagcgagaacaaggagttcgcccgctgcctggccgccgaggacgtggtgttcatcggccccgacacccacgccatccaggccatgggcgacaagatcgagagcaagctgctggccaagaaggccgaggtgaacaccatccccggcttcgacggcgtggtgaaggacgccgaggaggccgtgcgcatcgcccgcgagatcggctaccccgtgatgatcaaggccagcgccggcggcggcggcaagggcatgcgcatcgcctgggacgacgaggagacccgcgacggcttccgcctgagcagccaggaggccgccagcagcttcggcgacgaccgcctgctgatcgagaagttcatcgacaacccccgccacatcgagatccaggtgctgggcgacaagcacggcaacgccctgtggctgaacgagcgcgagtgcagcatccagcgccgcaaccagaaggtggtggaggaggcccccagcatcttcctggacgccgagacccgccgcgccatgggcgagcaggccgtggccctggcccgcgccgtgaagtacagcagcgccggcaccgtggagttcctggtggacagcaagaagaacttctacttcctggagatgaacacccgcctgcaggtggagcaccccgtgaccgagtgcatcaccggcctggacctggtgcaggagatgatccgcgtggccaagggctaccccctgcgccacaagcaggccgacatccgcatcaacggctgggccgtggagtgccgcgtgtacgccgaggacccctacaagagcttcggcctgcccagcatcggccgcctgagccagtaccaggagcccctgcacctgcccggcgtgcgcgtggacagcggcatccagcccggcagcgacatcagcatctactacgaccccatgatcagcaagctgatcacctacggcagcgaccgcaccgaggccctgaagcgcatggccgacgccctggacaactacgtgatccgcggcgtgacccacaacatcgccctgctgcgcgaggtgatcatcaacagccgcttcgtgaagggcgacatcagcaccaagttcctgagcgacgtgtaccccgacggcttcaagggccacatgctgaccaagagcgagaagaaccagctgctggccatcgccagcagcctgttcgtggccttccagctgcgcgcccagcacttccaggagaacagccgcatgcccgtgatcaagcccgacatcgccaactgggagctgagcgtgaagctgcacgacaaggtgcacaccgtggtggccagcaacaacggcagcgtgttcagcgtggaggtggacggcagcaagctgaacgtgaccagcacctggaacctggccagccccctgctgagcgtgagcgtggacggcacccagcgcaccgtgcagtgcctgagccgcgaggccggcggcaacatgagcatccagttcctgggcaccgtgtacaaggtgaacatcctgacccgcctggccgccgagctgaacaagttcatgctggagaaggtgaccgaggacaccagcagcgtgctgcgcagccccatgcccggcgtggtggtggccgtgagcgtgaagcccggcgacgccgtggccgagggccaggagatctgcgtgatcgaggccatgaagatgcagaacagcatgaccgccggcaagaccggcaccgtgaagagcgtgcactgccaggccggcgacaccgtgggcgagggcgacctgctggtggagctggagtgagcggccgcagtactgctagcactagtattggatcggatcccgggcccgtcgactgcagaggcctgcatgcaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 4025 bp, Artificial
  synthetic oligonucleotide
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggacgaattcgagctcggtacctcgcgaatgcatctagatccaatagatctgacgatggccggcttctgggtgggcaccgcccccctggtggccgccggccgccgcggccgctggcccccccagcagctgatgctgagcgccgccctgcgcaccctgaagcacgtgctgtactacagccgccagtgcctgatggtgagccgcaacctgggcagcgtgggctacgaccccaacgagaagaccttcgacaagatcctggtggccaaccgcggcgagatcgcctgccgcgtgatccgcacctgcaagaagatgggcatcaagaccgtggccatccacagcgacgtggacgccagcagcgtgcacgtgaagatggccgacgaggccgtgtgcgtgggccccgcccccaccagcaagagctacctgaacatggacgccatcatggaggccatcaagaagacccgcgcccaggccgtgcaccccggctacggcttcctgagcgagaacaaggagttcgcccgctgcctggccgccgaggacgtggtgttcatcggccccgacacccacgccatccaggccatgggcgacaagatcgagagcaagctgctggccaagaaggccgaggtgaacaccatccccggcttcgacggcgtggtgaaggacgccgaggaggccgtgcgcatcgcccgcgagatcggctaccccgtgatgatcaaggccagcgccggcggcggcggcaagggcatgcgcatcgcctgggacgacgaggagacccgcgacggcttccgcctgagcagccaggaggccgccagcagcttcggcgacgaccgcctgctgatcgagaagttcatcgacaacccccgccacatcgagatccaggtgctgggcgacaagcacggcaacgccctgtggctgaacgagcgcgagtgcagcatccagcgccgcaaccagaaggtggtggaggaggcccccagcatcttcctggacgccgagacccgccgcgccatgggcgagcaggccgtggccctggcccgcgccgtgaagtacagcagcgccggcaccgtggagttcctggtggacagcaagaagaacttctacttcctggagatgaacacccgcctgcaggtggagcaccccgtgaccgagtgcatcaccggcctggacctggtgcaggagatgatccgcgtggccaagggctaccccctgcgccacaagcaggccgacatccgcatcaacggctgggccgtggagtgccgcgtgtacgccgaggacccctacaagagcttcggcctgcccagcatcggccgcctgagccagtaccaggagcccctgcacctgcccggcgtgcgcgtggacagcggcatccagcccggcagcgacatcagcatctactacgaccccatgatcagcaagctgatcacctacggcagcgaccgcaccgaggccctgaagcgcatggccgacgccctggacaactacgtgatccgcggcgtgacccacaacatcgccctgctgcgcgaggtgatcatcaacagccgcttcgtgaagggcgacatcagcaccaagttcctgagcgacgtgtaccccgacggcttcaagggccacatgctgaccaagagcgagaagaaccagctgctggccatcgccagcagcctgttcgtggccttccagctgcgcgcccagcacttccaggagaacagccgcatgcccgtgatcaagcccgacatcgccaactgggagctgagcgtgaagctgcacgacaaggtgcacaccgtggtggccagcaacaacggcagcgtgttcagcgtggaggtggacggcagcaagctgaacgtgaccagcacctggaacctggccagccccctgctgagcgtgagcgtggacggcacccagcgcaccgtgcagtgcctgagccgcgaggccggcggcaacatgagcatccagttcctgggcaccgtgtacaaggtgaacatcctgacccgcctggccgccgagctgaacaagttcatgctggagaaggtgaccgaggacaccagcagcgtgctgcgcagccccatgcccggcgtggtggtggccgtgagcgtgaagcccggcgacgccgtggccgagggccaggagatctgcgtgatcgaggccatgaagatgcagaacagcatgaccgccggcaagaccggcaccgtgaagagcgtgcactgccaggccggcgacaccgtgggcgagggcgacctgctggtggagctggagtgagcggccgcagtactgctagcactagtattggatcggatcccgggcccgtcgactgcagaggcctgcatgcaagcttgcctcgagcagcgctgctcgagagatctacgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttgtaggtaaccacgtgcggaccgagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 9 - DNA, 1227 bp, Homo sapiens
atgcaggtcaccctgaagaccctccagcagcagaccttcaagatagacattgaccccgaggagacggtgaaagcactgaaagagaagattgaatctgaaaaggggaaagatgcctttccagtagcaggtcaaaaattaatttatgcaggcaaaatcctcaatgatgatactgctctcaaagaatataaaattgatgagaaaaactttgtggtggttatggtgaccaaacccaaagcagtgtccacaccagcaccagctacaactcagcagtcagctcctgccagcactacagcagttacttcctccaccaccacaactgtggctcaggctccaacccctgtccctgccttggcccccacttccacacctgcatccatcactccagcatcagcgacagcatcttctgaacctgcacctgctagtgcagctaaacaagagaagcctgcagaaaagccagcagagacaccagtggctactagcccaacagcaactgacagtacatcgggtgattcttctcggtcaaacctttttgaagatgcaacgagtgcacttgtgacgggtcagtcttacgagaatatggtaactgagatcatgtcaatgggctatgaacgagagcaagtaattgcagccctgagagccagtttcaacaaccctgacagagcagtggagtatcttttaatgggaatccctggagatagagaaagtcaggctgtggttgacccccctcaagcagctagtactggggctcctcagtcttcagcagtggctgcagctgcagcaactacgacagcaacaactacaacaacaagttctggaggacatccccttgaatttttacggaatcagcctcagtttcaacagatgagacaaattattcagcagaatccttccttgcttccagcgttactacagcagataggtcgagagaatcctcaattacttcagcaaattagccaacaccaggagcattttattcagatgttaaatgaaccagttcaagaagctggtggtcaaggaggaggaggtggaggtggcagtggaggaattgcagaagctggaagtggtcatatgaactacattcaagtaacacctcaggaaaaagaagctatagaaaggttaaaggcattaggatttcctgaaggacttgtgatacaagcgtattttgcttgtgagaagaatgagaatttggctgccaattttcttctacagcagaactttgatgaagat

SEQ ID NO: 10 - protein, 409 aa, Homo sapiens
MQVTLKTLQQQTFKIDIDPEETVKALKEKIESEKGKDAFPVAGQKLIYAGKILNDDTALKEYKIDEKNFVVVMVTKPKAVSTPAPATTQQSAPASTTAVTSSTTTTVAQAPTPVPALAPTSTPASITPASATASSEPAPASAAKQEKPAEKPAETPVATSPTATDSTSGDSSRSNLFEDATSALVTGQSYENMVTEIMSMGYEREQVIAALRASFNNPDRAVEYLLMGIPGDRESQAVVDPPQAASTGAPQSSAVAAAAATTTATTTTTSSGGHPLEFLRNQPQFQQMRQIIQQNPSLLPALLQQIGRENPQLLQQISQHQEHFIQMLNEPVQEAGGQGGGGGGGSGGIAEAGSGHMNYIQVTPQEKEAIERLKALGFPEGLVIQAYFACEKNENLAANFLLQQNFDED